(19) United States
(12) Patent Application Publication
(10) Pub. No.: US 2026/0191489 A1
(43) Pub. Date: Jul. 9, 2026

(54) SYSTEM AND METHOD FOR BONE FRACTURE RISK ASSESSMENT

(71) Applicant: Diagno Intelligent Systems Private Limited, Chennai, (IN)

(72) Inventors: Felix Enigo Varghese Sicily, Chennai (IN); Anburajan Mariamichael, Chennai (IN); Mary Natasha Anburajan, Chennai (IN)

(73) Assignee: Diagno Intelligent Systems Private Limited, Chennai (IN)

(21) Appl. No.: 19/132,458

(22) PCT Filed: Nov. 23, 2023
(86) PCT No.: PCT/IN2023/051083

§ 371 Date: May 23, 2025

(30) Foreign Application Priority Data
Nov. 23, 2022 (IN) 202241067402

(51) Int. Cl. A61B 6/50; A61B 6/00

(52) U.S. Cl. CPC A61B 6/505; A61B 6/482

(57) ABSTRACT

The present invention relates to a system and a method for bone fracture risk assessment and provides for a system to predict Osteoporosis and Osteopenia and its associated fracture risk automatically from conventional chest X-ray image. The invention provides an automated digital X-ray bilateral Clavicle Radiogrammetry and its estimation of Cortical Thickness and Calculation of Cortical bone mass indices of Clavicle to predict dual Hips (Neck) Bone Mineral Density (BMD), dual Hips (Total) Bone Mineral Density (BMD) and Spine (Total) Bone Mineral Density (BMD) as measured by dual energy X-ray absorptiometry (DXA) bone densitometer and 10-year probability of both Major Bones (hip, spine, humerus or forearm)—and Hip-Osteoporotic Fracture Risk Scores. Using the same chest X-ray, an automated prediction of Osteoporosis and Osteopenia at both dual hips and spine regions and 10-year probability of both Major Bones and Hip Osteoporotic Fracture Risk using Artificial Intelligence (AI).

### RELATED PATENT APPLICATION(S)

This application claims the priority to and benefit of Indian Provisional Patent Application No. 202241067402 filed on Nov. 23, 2022; the disclosures of which are incorporated herein by reference.

### FIELD OF THE INVENTION

The present invention generally relates to the field of risk assessment systems. Particularly, the present invention relates to system and method for osteoporotic bone fracture risk assessment. More particularly, the invention relates to development of system and method for osteoporotic bone fracture risk assessment using novel innovative approaches to predict Osteoporosis and Osteopenia and its associated future fracture risk automatically with high accuracy from a conventional chest X-ray image.

### BACKGROUND OF THE INVENTION

Prevention of osteoporotic fractures with consequent reductions in healthcare costs and excess morbidity and mortality is an important clinical goal. Conventional techniques may not fulfil availability, cost factor and non-portable nature of bone risk assessment machines and this may lead to inaccessibility of these bone risk assessment machines to majority of older women and men in rural and suburban areas. The conventional techniques are capable of measuring Speed of Sound (SOS) and Broadband Ultrasound Attenuation (BUA) at heel bone and may not measure Bone Mineral Density (BMD) at either hip or spine. Further, the Peripheral Dual energy X-ray Absorptiometry (pDXA) bone densitometer may measure the BMD at forearm and heel bone and may not measure the BMD at either the hip or the spine. Further, these Peripheral Dual energy X-ray Absorptiometry (pDXA) is operator dependent and are moderately accurate.

Further, existing screening tools for prediction of Osteoporosis & its associated fracture risk are based on scores calculated from patient's clinical risk factors and with/without the patient's hip (neck) BMD measured by the Central Dual energy X-ray Absorptiometry (cDXA) Bone Densitometer. These existing screening tools use simple questionnaires, which assess a complex of various risk factors limited to advancing age of the patient, body weight, use of glucocorticoids, or hormone replacement therapy to compute the bone risk assessment.

U.S. Pat. No. 9,532,761B2 discloses a method and system for quantitatively evaluating bone fracture risk in a living being that generate a value for an index indicative of a degree of bone fracture risk. The method of prior art includes the step of acquiring values for a height H, a weight W and a measured bone mineral density BMDof the living being. The method further includes the step of calculating a quantitative bone fracture risk index QI associated with the living being in accordance with the formula QI=H^{α}*W^{β}P/BMD^{γ} where α, β, and γ are constants selected based on previously obtained data indicative of bone fracture risk.

U.S. Pat. No. 7,801,347B2 discloses methods and systems for computer assisted detection of arterial calcification, for example in the abdominal artery, by using measurements such as those conventionally taken with a bone densitometer at single energy or dual energy, or by a computed tomography, CT/quantitative computed tomography, QCT device for a localization of scout view, and for using the calcification assessment either alone or with other information to assess and report a risk of a cardiovascular event, alone or together with other information such as BMD and vertebral fracture risk.

However, all these systems and methods may use clinical risk factors and may not measure Bone Mineral Density (BMD) values by Dual Energy X-ray Absorptiometry (DXA) and may be moderately accurate in predicting future bone fracture risk. A detailed research literature survey reveals that there is no digital X-ray image based automated estimation of both Hip- and Spine-BMD with good accuracy using an automated digital X-ray bilateral clavicle radiogrammetry and an automated estimation of 10-year probability of both major bones (hip, spine, humerus or forearm)- and hip-osteoporotic fracture risk using an artificial intelligence (AI) with machine learning (ML) and deep learning (DL) techniques for screening the risk of osteoporosis.

Therefore, there is need for development of system and method for osteoporotic bone fracture risk assessment to predict Osteoporosis and Osteopenia and its associated fracture risk automatically with high accuracy from a conventional chest X-ray image using novel innovative approaches.

Thus, the present invention relates to an Artificial Intelligence (AI) with machine learning (ML) or deep learning (DL) techniques-based tool indigenously configured to estimate hip Bone Mineral Density (BMD) as well as spine Bone Mineral Density (BMD) and 10-year probability of both major bones- and hip-osteoporotic fracture risk scores by an automatic way using the digital or computed chest radiograph which may be tested at multisite centers across country, where there may be a high incidence of Osteoporosis in local population, exorbitant healthcare costs or limited access to state-of-the art imaging centers. In particular, the present invention aims to design and extend routine chest X-ray (radiograph) investigation to serve as a means to assess bone health of an individual quantitatively in a resource constrained environment.

### OBJECT(S) OF THE INVENTION

A primary object of the present invention is to develop a system for bone fracture risk assessment.

Another object of the present invention is to develop an Artificial Intelligence (AI)-powered system or tool to predict Osteoporosis and Osteopenia and its associated fracture risk automatically with high accuracy from conventional chest X-ray image using novel innovative approaches.

Another object of the present invention is to provide a method for performing risk assessment of bone fracture using a bone fracture risk assessment system.

Another object of the present invention is to provide a method for performing risk assessment of bone fracture using numerical and non-numerical approaches.

Another object of the present invention is to provide a method for an automated digital X-ray bilateral Clavicle Radiogrammetry and its estimation of Cortical Thickness of the Clavicle and Calculation of Cortical bone mass indices of the Clavicle to predict Hip (Neck) Bone Mineral Density (BMD), Hip (Total) BMD and Spine (Total) BMD and its associated 10-year probability of Major Bones- and Hip-Osteoporotic Fracture Risk Scores with high accuracy from a Low-cost Chest X-ray image.

Another object of the present invention is to provide a method for an automated prediction of Osteoporosis and Osteopenia at dual hips and spine using Artificial Intelligence (AI) with automated classifications as per World Health Organization (WHO)'s Diagnostic Criteria in machine learning (ML) model and/or deep learning (DL) model and/or ensembling model with high accuracy from a chest X-ray image.

Another object of the present invention is to provide a method for an automated prediction of 10-year probability of major bones (hip, spine, humerus or forearm) Osteoporotic fracture risk and hip fracture risk using Artificial Intelligence (AI) with automated classifications in machine learning (ML) model and/or deep learning (DL) model and/or ensembling model with high accuracy from a chest X-ray image.

Another object of the present invention is to design and extend routine chest X-ray (radiograph) investigation to serve as a means to an automated assessment of bone health of an individual quantitatively in a resource constrained environment.

Yet another object of the present invention is to develop an ‘iOsteoporos Screen’ an Intelligent Osteoporosis Screening Tool using a conventional chest X-ray image, which is cost-effective and is readily available Tool to spot out older women and men, who are at the risk for osteoporosis & osteopenia at dual hips and spine and its associated future bone fracture risk

### SUMMARY OF THE INVENTION

Accordingly, the present invention provides a system and a method for bone fracture risk assessment. Particularly, the present invention discloses a system and a method for bone fracture risk assessment to predict Osteoporosis and Osteopenia and its associated fracture risk automatically with high accuracy from a conventional chest X-ray image using novel innovative approaches.

More particularly, the present invention relates to a method for performing risk assessment of bone fracture using numerical and non-numerical approaches. Firstly, an automated digital X-ray bilateral Clavicle Radiogrammetry and its measurement of Cortical Thickness and Cortical Width of the Clavicle and Calculation of Cortical bone mass indices of the Clavicle to predict Hip (Neck) Bone Mineral Density (BMD), Hip (Total) BMD and Spine (Total) BMD as measured by dual energy X-ray absorptiometry (DXA) bone densitometer and its associated 10-year probability of Major Bone and Hip Osteoporotic Fracture Risk Scores as calculated by FRAX Tool with high accuracy from a Low-cost Chest X-ray image is provided as numerical approach in the present invention. Further, in non-numerical approach, an automated prediction of Osteoporosis and Osteopenia at dual hips and spine as per World Health Organization (WHO)'s Diagnostic Criteria and 10-year probability of major bones (hip, spine, humerus or forearm) Osteoporotic fracture risk and hip fracture risk using Artificial Intelligence (AI) with automated classifications in machine learning (ML) model and/or deep learning (DL) model and/or ensembling model with high accuracy from a digital or computed chest X-ray image is provided.

Thus, the present invention relates to an Artificial Intelligence (AI) with machine learning (ML) or deep learning (DL) techniques-based tool indigenously configured to estimate bilateral hip Bone Mineral Density (BMD) by an automatic way using the digital or computed chest radiograph which may be tested at multisite centers across country, where there may be a high incidence of Osteoporosis in local population, exorbitant healthcare costs or limited access to state-of-the art imaging centers. The present invention aims to design and extend routine chest X-ray (radiograph) investigation to serve as a means to assess bone health of an individual quantitatively in a resource constrained environment.

In one aspect of the present invention, the invention provides a system (101) for bone fracture risk assessment, wherein the system (101) comprises:
a processor or a central processing unit (CPU) (102);
an Input/Output (I/O) interface (103) coupled with the processor (102); and
a memory (104) for storing instructions executable by the processor (102), wherein the memory (104) comprises modules (106) and data (105), wherein the data (105) comprises one or more images (107), region of interests (108) and a bone mineral density (BMD) (109), and wherein the modules (106) comprise a receiving module (110), a determining module (111), a Bone Mineral Density (BMD) estimation module (112), a risk assessment module (113) and other modules (114).

The modules (106) are configured to perform the estimation of bone fracture risk employing the data (105).

The receiving module (110) is configured to receive the one or more images (107) of a standard digital or computed Chest X-ray radiograph.

The one or more images (107) comprises the standard digital or computed chest X-ray radiograph for a fully automated computerized digital or computed X-ray radiographic image processing and one or more images (107) is in a form of a gray-scale image.

The determining module (111) is configured to:
determine Region of Interests (ROI) (108) on the one or more images (107); and
utilize a deep neural network architecture and the deep neural network architecture is trained with a data set of images and their corresponding masks created at the Region of Interests (ROI) (108),
wherein the determining module (111) performs a mapping process to segment masked region (clavicle bone) automatically from the one or more images (107) using the trained deep neural network architecture.

The Region of Interests (ROI) (108) is an intersection point of the bilateral clavicle bone and an end enclosure part of a rib bone being an ideal location for an automated digital X-ray radiographic bilateral clavicle radiogrammetry measurements.

The Bone Mineral Density (BMD) estimation module (112) is configured to:
perform automated digital or computed X-ray radiographic bilateral clavicle radiogrammetry, and
estimate a Hip (NECK) BMD (109) in g/cm2, Hip (TOTAL) BMD (109) in g/cm2, Spine (TOTAL) BMD (109) in g/cm2, 10-year probability of Major Bones (Hip, Spine, Humerus or forearm) Osteoporotic Fracture Risk Score (%) and 10-year probability of Osteoporotic Hip Fracture Risk Score (%) by employing the calculated Bone Mass Indices of the Clavicle Region of Interests (ROI) (108).

The risk assessment module (113) is configured to output a risk assessment based on calculated T-Score of Hip (Neck), Hip (Total) and Spine (Total) from the estimated bone mineral density (BMD) (109) values of the same by comparing the calculated T-score of Hip (Neck), Hip (Total) and Spine (Total) to a cutoff T-score values as per World Health Organization (WHO)'s Diagnostic Criteria and predicting whether the patient is Normal, having Osteopenia and Osteoporosis.

The risk assessment module (113) is configured to output a risk assessment based on estimated fracture risk scores by comparing the estimated fracture risk scores with published threshold values comprising 10-year probability of Major Bones Osteoporotic Fracture Risk Score (%)≥10 and 10-year probability of Osteoporotic Hip Fracture Risk Score (%)≥3 and determining the high future risk for Osteoporotic fracture.

The other modules (114) are configured to perform a standard pre-processing on the one or more images (107), and
wherein the standard pre-processing is performed by employing an image equalization technique to obtain the one or more images (107) with improved contrast.

In another aspect, the invention provides a method for bone fracture risk assessment employing a system (101), wherein the method comprises the steps:
(a) receiving, by a receiving module (110) one or more images (107);
(b) performing a standard pre-processing on the one or more images (107);
(c) determining, by a determining module (111) Region of Interests (ROI) (108) on the one or more images (107);
(d) obtaining a plurality of measurements for an accurate automated digital or computed X-ray radiographic bilateral clavicle radiogrammetry;
(e) performing, by the bone mineral density (BMD) estimation module (112) the automated digital or computed X-ray radiographic bilateral clavicle radiogrammetry employing both the measurements of cortical thickness and cortical width of the clavicle obtained at step (d);
(f) estimating, by the bone mineral density (BMD) estimation module (112) Cortical Bone Mass Indices of the Clavicle;
(g) estimating, by the bone mineral density (BMD) estimation module (112) a bone mineral density (BMD) (109) in g/cm2 employing the estimated bone mass indices; and
(h) outputting, by a risk assessment module (113) a risk assessment based on the estimated bone mineral density (BMD) (109).

In said method, the one or more images (107) comprises the standard digital or computed chest X-ray radiograph for a fully automated computerized digital or computed X-ray radiographic image processing and one or more images (107) is in a form of a gray-scale image.

The Region of Interests (ROI) (108) is bilateral clavicle bones being masked employing an Image Annotator for an automated computerized accurate segmentation of the Region of Interests (ROI) (108) employing UNet a deep neural architecture that performs semantic segmentation in a complex environment.

The step (c) comprises performing, by the other modules (114) post processing techniques, comprising a Contrast Limited Adaptive Histogram Equalization (CLAHE), a blur, a dilation, a median filter, a scar operator, a binarization technique, connected component analysis and morphological operations, a Two-Dimensional (2D) filter on the Region of Interests (ROI) (108).

The step (c) comprises training, by the determining module (111) a deep neural network architecture with a data set of images and their corresponding masks created at the Region of Interests (ROI) (108).

The step (c) comprises performing, by the determining module (111) a mapping process to segment the masked region (clavicle bone) automatically from the one or more images (107) employing the trained deep neural network architecture.

The Region of Interests (ROI) (108) is an intersection point of the bilateral clavicle bone and an end enclosure part of a rib bone being an ideal location for an automated digital or computed X-ray radiographic bilateral clavicle radiogrammetry measurements, wherein there are no overlapping or surrounding bone structures, soft tissues, and provides high reproducibility for the accurate automated digital or computed X-ray radiographic bilateral clavicle radiogrammetry measurements.

The step (d) comprises utilizing, by the determining module (111) a centroid region at the Region of Interests (ROI) (108) of the bilateral clavicle bone as a starting point and proceeding towards an upper direction by incrementing pixel coordinates till a white pixel is determined and designating the white pixel as the starting point of bone (1); progressing, by the determining module (111) the centroid region of the bilateral clavicle bone in an upper direction till a black pixel is determined, and designating the black pixel as an outer point of the bilateral clavicle bone (2); and repeating the same approach in a downward direction by decrementing the pixel coordinates from the centroid region of the bilateral clavicle bone, and subsequently (3) and (4) are determined.

In step (e), the measurements comprise points 1, 2, 3, 4 of bone to determine Endosteal width, d (cm) and Periosteal width, D (cm).

In step (f), the bone mass indices are estimated by employing the obtained Endosteal width, d and Periosteal width, D at step (e).

In step (g), a Hip (NECK) BMD (109) in g/cm2, Hip (TOTAL) BMD (109) in g/cm2, Spine (TOTAL) BMD (109) in g/cm2, 10-year probability of Major Bones (Hip, Spine, Humerus or forearm) Osteoporotic Fracture Risk Score (%) and 10-year probability of Osteoporotic Hip Fracture Risk Score (%) by employing the measured Cortical Thickness of the Clavicle and the calculated Bone Mass Indices of the Clavicle Region of Interests (ROI) (108).

In step (h), the risk assessment based on the calculated T-score of Hip (Neck), Hip (Total) and Spine (Total) from the estimated bone mineral density (BMD) (109) values of Hip (Neck), Hip (Total), and Spine (Total) are outputted by comparing the calculated T-scores of Hip (Neck), Hip (Total) and Spine (Total)) to a Cutoff T-score values as per World Health Organization (WHO)'s Diagnostic Criteria and predicting whether the patient is Normal, having Osteopenia and Osteoporosis.

In step (h), the risk assessment based on estimated fracture risk scores is outputted by comparing the estimated fracture risk scores with published threshold values comprising 10-year probability of Major Bones Osteoporotic Fracture Risk Score (%)≥10 and 10-year probability of Osteoporotic Hip Fracture Risk Score (%)≥3 and determining the high future risk for Osteoporotic fracture.

The above said method is employed for an automated estimation of Cortical Thickness of the Clavicle and Calculation of Cortical bone mass indices of the Clavicle to predict Hip (Neck) Bone Mineral Density (BMD), Hip (Total) BMD and Spine (Total) BMD and its associated 10-year probability of Major Bone and Hip Osteoporotic Fracture Risk scores with high accuracy from a Low-cost Chest X-ray image using an automated digital X-ray radiographic bilateral clavicle radiogrammetry.

In another aspect, the present inventio provides a method for bone fracture risk assessment employing a system (101), wherein the method comprises the steps:
(a) obtaining one or more images (107) as input by a receiving module (110);
(b) performing a standard pre-processing on the one or more images (107) by other modules (114);
(c) performing an automated segmentation of region of interests (ROI) on the one or more images (107);
(d) applying, augmentation techniques to increase number of images dataset and to generalize the model performance to handle versatile images;
(e) building and deployment of multiple Deep Learning (DL) models;
(f) validating and testing of multiple Deep Learning (DL) models;
(g) selecting the best Deep Learning (DL) model in each binary classification category based on the model's validation and test data performance;
(h) obtaining the best Deep Learning (DL) model;
(i) extracting deep features from the penultimate layer (dense layer) of the best Deep Learning (DL) model;
(j) applying different feature selection techniques to the datasets for selection of Deep Learning (DL) features;
(k) employing various Machine Learning (ML) classifiers with various combination of feature selection techniques;
(l) creating the separate machine learning (ML) models for the different categories;
(m) validating and testing of multiple machine learning (ML) models;
(n) selecting the best machine learning (ML) model of each category based on the performance of model on the validation and test dataset; and
(o) computing final impression by ensembling the results obtained from combination of both deep learning (DL) and machine learning (ML) approaches.

In step (a), the one or more images (107) comprises a standard digital Chest X-ray for an automated computerized image processing. The one or more images (107) may be in DICOM, jpg, png or tiff format.

At step (b), standard pre-processing is performed using an image equalization technique to obtain the one or more images (107) with improved contrast; and the step (b) comprises:
checking the image format and if the image is in DICOM format, automatically extracting patient information, including Name, Age, and Sex, and saving it in the report;
converting, the received image into PNG format to ensure uniformity for all types of images in the subsequent procedures;
verifying, if the received image is a CXR using GoogleNet classifier model, if not, discarding the image and requesting an upload of a chest X-ray image; and
resizing the image into 512×512 size and applying Contrast Limited Adaptive Histogram Equalization (CLAHE) to the resized input image.

Iin step (c), an automated Region of Interests (ROI) segmentation is performed wherein EfficientNet B7 with Unet decoder and 40 dropout is trained and obtained the model to automatically segment the Region of Interests (ROI) (108) from the Contrast Limited Adaptive Histogram Equalization (CLAHE) applied image.

The Region of Interests (ROI) (108) is a square region on CXR covering clavicle bone end at the left and right side, extend top up to shoulder neck interaction at the top and cover up to L2 at bottom end.

In step (d), the model is well trained and evaluated with parameters such as dice_coef, dice_loss, IoU, Recall, and Precision and the grayscale image information of the automatically segmented Region of Interests (ROI) (108) is extracted by mapping Region of Interests (ROI) mask with original grayscale image.

In step (e), the different deep learning models are selected to perform automatic classification task and each model to classify all categories are trained with different hyperparameters combinations; and
again, augmentation techniques are applied to increase number of images dataset and to generalize the classification model performance to handle versatile input images.

In step (l), all the machine learning (ML) models are trained with all combinations of feature selection techniques.

The above said method is employed for an automated prediction of Osteoporosis and Osteopenia by employing Artificial Intelligence (AI) with automated classifications in machine learning (ML) model and/or deep learning (DL) model and/or ensembling model with high accuracy from a chest X-ray image.

In another aspect, the invention provides a method for bone fracture risk assessment employing a system (101), wherein the method comprises the steps:
(a) calculating FRAX Scores;
(b) obtaining one or more images (107) as input by a receiving module (110);
(c) performing a standard pre-processing on the one or more images (107) by other modules (114);
(d) performing an automated segmentation of region of interests (ROI) on the one or more images (107);
(e) applying, augmentation techniques to increase number of images dataset and to generalize the model performance to handle versatile images;
(f) building and deployment of multiple Deep Learning (DL) models;
(g) validating and testing of multiple Deep Learning (DL) models;
(h) selecting the best Deep Learning (DL) model in each binary classification category based on the model's validation and test data performance; and
(i) computing the results of Deep Learning (DL) classification by “Ensemble” method.

The above said method is employed for an automated prediction of 10-year probability of Major Bone (Hip, Spine, Humerus or Forearm) Osteoporotic Fracture Risk and Hip Osteoporotic Fracture Risk from conventional chest x-ray image by ensembling the Deep Learning (DL) models for 10-Year Probability of Major Bones Osteoporotic Fracture Risk Score and 10-Year Probability of Hip Osteoporotic Fracture Risk Score.

In another aspect, the present invention provides a method for bone fracture risk assessment employing a system (101), wherein the method comprises the steps:
(a) obtaining the best Deep Learning (DL) model;
(b) extracting deep features from the penultimate layer (dense layer) of the best Deep Learning (DL) model;
(c) applying different feature selection techniques to the datasets for selection of Deep Learning (DL) features;
(d) employing various Machine Learning (ML) classifiers with various combination of feature selection techniques;
(e) creating the separate machine learning (ML) models for the different categories;
(f) validating and testing of multiple machine learning (ML) models;
(g) selecting the best model of each category based on the performance of model on the validation and test dataset; and
(h) computing the results of Machine Learning (ML) classification by “Ensemble” method.

The above said method is employed for an automated prediction of 10-year probability of Major Bone (Hip, Spine, Humerus or Forearm) Osteoporotic Fracture Risk and Hip Osteoporotic Fracture Risk from conventional chest x-ray image by ensembling the Machine Learning (DL) models for 10-Year Probability of Major Bones Osteoporotic Fracture Risk Score and 10-Year Probability of Hip Osteoporotic Fracture Risk Score.

The above description merely is an outline of the technical solution of the present disclosure. The summary is descriptive and exemplary only and is not intended to be in any way restricting. In order to know the technical means of the present disclosure more clearly so that implementation may be carried out according to contents of the specification, and in order to make the above and other objectives, characteristics and advantages of the present disclosure clear and easy to understand, specific embodiments of the present invention will be described in detail below.

### BRIEF DESCRIPTION OF THE DRAWINGS

The embodiments of the disclosure itself, as well as a preferred mode of use, further objectives and advantages thereof, will best be understood by reference to the following detailed description of an illustrative embodiment when read in conjunction with the accompanying drawings. One or more embodiments are now described, by way of example only, with reference to the accompanying drawings in which:

FIG. 1 illustrates an exemplary architecture of a bone fracture risk assessment system, in accordance with some embodiments of the present disclosure.

FIG. 2 (a) shows an exemplary flow chart illustrating the steps of numerical approach for an automated estimation of Cortical Thickness of the Clavicle and Calculation of Cortical bone mass indices of the Clavicle to predict Hip (Neck) Bone Mineral Density (BMD), Hip (Total) BMD and Spine (Total) BMD and its associated 10-year probability of Major Bone and Hip Osteoporotic Fracture Risk scores with high accuracy from a Low-cost Chest X-ray image using an automated bilateral digital X-ray radiographic clavicle radiogrammetry in accordance with some embodiments of the present disclosure.

FIG. 2 (b) shows an exemplary flow chart illustrating a method for performing risk assessment of bone fracture using an automated digital X-ray radiographic bilateral clavicle radiogrammetry, in accordance with some embodiments of the present disclosure.

FIG. 3 (a-c) shows a conventional Digital Chest X-ray of the patient including segmented images of it.

FIG. 4 (a-b) shows total 10 selected regions for measurements M1-M10 of an automated digital X-ray radiographic bilateral (both left side and right side) Clavicle Radiogrammetry.

FIG. 5 shows an exemplary histogram for one of the regions of measurements M1-M10 of an automated digital X-ray radiographic bilateral (both left side and right side) Clavicle Radiogrammetry.

FIG. 6 (a-d) shows comparative graphs for Score 1: prediction of hip (neck) bone mineral density (BMD) using an automated digital X-ray radiographic clavicle radiogrammetry (from a low-cost chest x-ray image) using an artificial neural network (ANN) regression model with high correlation coefficient: (a)-Regression Model: Actual vs. Predicted; (b)-Residual Plot; (c)-Distribution of Residuals; and (d)-QQ Plot.

FIG. 7 (a-d) shows comparative graphs for Score 2: prediction of hip (total) bone mineral density (BMD) using an automated digital X-ray radiographic clavicle radiogrammetry (from a low-cost chest x-ray image) using a pattern recognition neural network (PNNN) regression model with high correlation coefficient: (a)-Regression Model: Actual vs. Predicted; (b)-Residual Plot; (c)-Distribution of Residuals; and (d) QQ Plot.

FIG. 8 (a-d) shows comparative graphs for Score 3: prediction of spine (total) bone mineral density (BMD) using an automated digital X-ray radiographic clavicle radiogrammetry (from a low-cost chest x-ray image) using an artificial neural network (ANN) regression model with high correlation coefficient: (a)-Regression Model: Actual vs. Predicted; (b)-Residual Plot; (c)-Distribution of Residuals; and (d)-QQ Plot.

FIG. 9 (a-d) shows comparative graphs for Score 4: prediction of 10-year probability of Major Bones (hip, spine, humerus or forearm) Osteoporotic Fracture Risk Score (FRAX Score) from an automated digital X-ray radiographic clavicle radiogrammetry (from a low-cost chest x-ray image) using pattern recognition neural network (PRNN) regression model with high correlation coefficient: (a)-Regression Model: Actual vs. Predicted; (b)-Residual Plot; (c)-Distribution of Residuals; and (d)-QQ Plot.

FIG. 10 (a-d) shows comparative graphs for Score 5: prediction of 10-year probability of Osteoporotic Hip Fracture Risk Score (FRAX Score) from an automated digital X-ray radiographic clavicle radiogrammetry (from a low-cost chest x-ray image) using radial basis neural network (RBRM) regression model with high correlation coefficient: (a)-Regression Model: Actual vs. Predicted; (b)-Residual Plot; (c)-Distribution of Residuals; and (d)-QQ Plot.

FIG. 11 (a) shows an exemplary flow chart illustrating the steps of non-numerical approach for an automated prediction of Osteoporosis and Osteopenia and 10-year probability of major bones (hip, spine, humerus or forearm) Osteoporotic fracture risk and hip fracture risk using Artificial Intelligence (AI) with automated classifications in machine learning (ML) model and/or deep learning (DL) model and/or ensembling model with high accuracy from a chest X-ray image.

FIG. 11 (b-c) shows an exemplary flow chart (300) illustrating a method for performing risk assessment of bone fracture using Artificial Intelligence (AI) with machine learning (ML) model and/or deep learning (DL) model and/or ensembling model with high accuracy from a chest X-ray image, in accordance with some embodiments of the present disclosure.

FIG. 12 shows schematic framework for deep learning (DL) approach for prediction of Osteoporosis at Hip and Spine from a conventional low-cost chest X-ray image.

FIG. 13 shows schematic framework for machine learning (ML) approach for prediction of Osteoporosis at Hip and Spine from a conventional low-cost chest X-ray image.

FIG. 14 shows schematic framework for the combination of both deep learning (DL) and machine learning (ML) approaches for prediction of Osteoporosis at Hip and Spine from a conventional low-cost chest X-ray image.

FIG. 15 (a-b) shows an exemplary flow chart (400) illustrating a method for performing risk assessment of bone fracture using a bone fracture risk assessment system with deep learning (DL) and machine learning (ML) approaches for prediction of 10-year probability of Major Bone and Hip Osteoporotic Fracture Risk from a conventional low-cost chest X-ray image, in accordance with some embodiments of the present disclosure.

FIG. 16 shows the schematic framework for deep learning (DL) approach for prediction of 10-year probability of Major Bone and Hip Osteoporotic Fracture Risk from a conventional low-cost chest X-ray image.

FIG. 17 shows the schematic framework for machine learning (ML) approach for prediction of 10-year probability of Major Bone and Hip Osteoporotic Fracture Risk from a conventional chest X-ray image.

### DETAILED DESCRIPTION OF THE INVENTION

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

While the disclosure is susceptible to various modifications and alternative forms, specific embodiment thereof has been shown by way of example in the drawings and will be described in detail below. It should be understood, however that it is not intended to limit the disclosure to the particular forms disclosed, but on the contrary, the disclosure is to cover all modifications, equivalents, and alternative falling within the spirit and the scope of the disclosure.

The terms “comprises”, “comprising”, or any other variations thereof, are intended to cover a non-exclusive inclusion, such that a setup, device or method that comprises a list of components or steps does not include only those components or steps but may include other components or steps not expressly listed or inherent to such setup or device or method. In other words, one or more elements in a device or system or apparatus proceeded by “comprises . . . a” does not, without more constraints, preclude the existence of other elements or additional elements in the device or system or apparatus.

In the following detailed description of the embodiments of the disclosure, reference is made to the accompanying drawings that form a part hereof, and in which are shown by way of illustration specific embodiments in which the disclosure may be practiced. These embodiments are described in sufficient detail to enable those skilled in the art to practice the disclosure, and it is to be understood that other embodiments may be utilized and that changes may be made without departing from the scope of the present disclosure. The following description is, therefore, not to be taken in a limiting sense.

The present invention provides a system and a method for bone fracture risk assessment.

Prevention of fractures with consequent reductions in healthcare costs and excess morbidity and mortality is an important clinical goal. Conventional techniques may not fulfil availability, cost factor and non-portable nature of bone risk assessment machines and this may lead to inaccessibility of these bone risk assessment machines to majority of older women and men in rural and suburban areas. The conventional techniques are capable of measuring Speed of Sound (SOS) and Broadband Ultrasound Attenuation (BUA) at heel bone and may not measure Bone Mineral Density (BMD) at either hip or spine. Further, the conventional techniques may measure the BMD at forearm and heel bone and may not measure the BMD at either the hip or the spine. Further, these conventional techniques are operator dependent and are moderately accurate.

Further, existing screening tools for prediction of Osteoporosis & its associated fracture risk are based on scores calculated from patient's clinical risk factors and with/without the patient's hip (neck) BMD measured by dual energy X-ray Absorptiometry (DXA) Bone Densitometer. These existing screening tools use simple questionnaires, which assess a complex of various risk factors limited to advancing age of the patient, body weight, use of glucocorticoids, or hormone replacement therapy to compute the bone risk assessment. All these tools may use clinical risk factors and may not measure Bone Mineral Density (BMD) values by Dual Energy X-ray Absorptiometry (DXA) and may be moderately accurate in predicting bone risk assessment. A detailed research literature survey reveals that there is no digital image based automated estimation of hip BMD with good accuracy using automated bilateral digital X-ray radiographic clavicle radiogrammetry and an automated estimation of 10-year probability of hip fracture risk using an artificial intelligence (AI) with machine learning (ML) and deep learning (DL) techniques for screening the risk of osteoporosis.

Accordingly, the present invention provides a system and a method for bone fracture risk assessment. Particularly, the present invention discloses a system and a method for bone fracture risk assessment to predict Osteoporosis and Osteopenia and its associated fracture risk automatically with high accuracy from a conventional chest X-ray image using novel innovative approaches.

More particularly, the present invention relates to a method for performing risk assessment of bone fracture using numerical and non-numerical approaches. Firstly, an automated estimation of Cortical Thickness of the Clavicle and Calculation of Cortical bone mass indices of the Clavicle to predict Hip (Neck) Bone Mineral Density (BMD), Hip (Total) BMD and Spine (Total) BMD and its associated 10-year probability of Major Bone and Hip Osteoporotic Fracture Risk scores with high accuracy from a Low-cost Chest X-ray image is provided as numerical approach in the present invention. Further, in non-numerical approach, an automated prediction of Osteoporosis and Osteopenia and 10-year probability of major bones (hip, spine, humerus or forearm) Osteoporotic fracture risk and hip fracture risk using Artificial Intelligence (AI) with automated classifications in machine learning (ML) model and/or deep learning (DL) model and/or ensembling model with high accuracy from a chest X-ray image is provided.

Thus, the present invention relates to an Artificial Intelligence (AI) with machine learning (ML) or deep learning (DL) techniques-based tool indigenously configured to estimate bilateral hip Bone Mineral Density (BMD) by an automatic way using the digital or computed chest radiograph which may be tested at multisite centers across country, where there may be a high incidence of Osteoporosis in local population, exorbitant healthcare costs or limited access to state-of-the art imaging centers. The present invention aims to design and extend routine chest X-ray (radiograph) investigation to serve as a means to assess bone health of an individual quantitatively in a resource constrained environment.

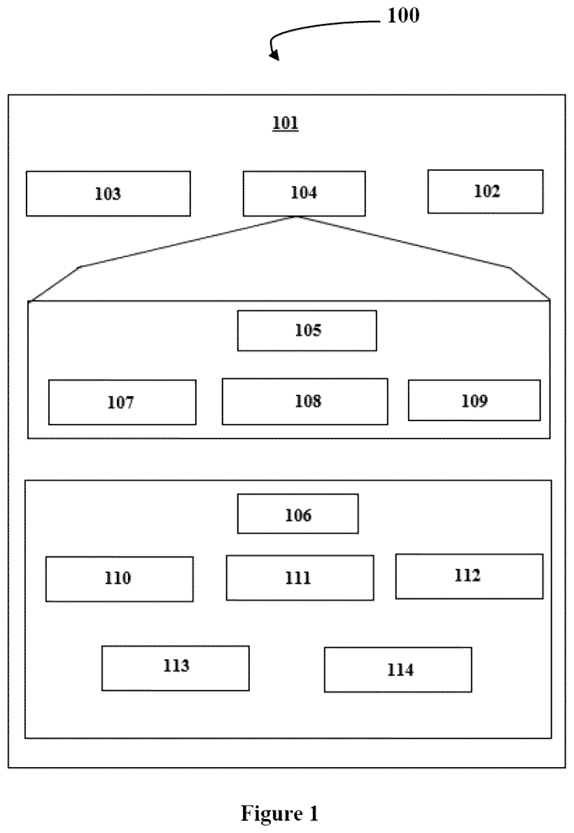

In one aspect, the present invention provides a system for bone fracture risk assessment. FIG. 1 illustrates an exemplary architecture (100) of a bone fracture risk assessment system (101), in accordance with the present disclosure.

In an exemplary embodiment of the present invention, the bone fracture risk assessment system (101) (also referred as risk assessment system) may be implemented in a variety of computing systems, such as a PACS (picture archiving and communication system), laptop, computer, a desktop computer, a Personal Computer (PC), a notebook, a smartphone, a tablet, e-book readers, a server, a network server, a cloud-based server and the like. The bone fracture risk assessment system (101) may include at least one Central Processing Unit (also referred to as “CPU” or “processor”) (102) and a memory (104) for storing instructions executable by the at least one processor (102). The at least one processor (102) may comprise at least one data processor for executing program components to execute user requests or system-generated requests. The memory (104) is communicatively coupled to the at least one processor (102). The memory (104) stores instructions, executable by the at least one processor (102), which, on execution, may cause the hip fracture risk assessment system (101) to perform the estimation, as disclosed in the present disclosure. In an embodiment, the memory (104) may include modules (106) and data (105). The modules (106) are configured to perform the steps of the present disclosure using the data (105) to perform the estimation. In an embodiment, each of the modules (106) may be a hardware unit which may be outside the memory (104) and coupled with the bone fracture risk assessment system (101). As used herein, the term modules (106) refer to an Application Specific Integrated Circuit (ASIC), an electronic circuit, a Field-Programmable Gate Arrays (FPGA), Programmable System-on-Chip (PSoC), a combinational logic circuit, and/or other suitable components that provide described functionality. The modules (106) when configured with the described functionality defined in the present disclosure will result in a novel hardware.

The bone fracture risk assessment system (101) (also referred as risk assessment system) further comprises an Input/Output (I/O) interface (103). The I/O interface (103) is coupled with the at least one processor (102) through which an input signal and/or an output signal is communicated. The input signal and the output signal may represent data received by the bone fracture risk assessment system (101) and data transmitted by the bone fracture risk assessment system (101), respectively. In an embodiment of the present disclosure, the bone fracture risk assessment system (101) may be configured to receive and transmit data via the I/O interface (103). The received data may comprise user inputs, and the like. The hip fracture risk assessment system (101) may communicate over a communication network. The communication network may include, without limitation, a direct interconnection, local area network (LAN), wide area network (WAN), wireless network (e.g., using Wireless Application Protocol), the Internet and the like.

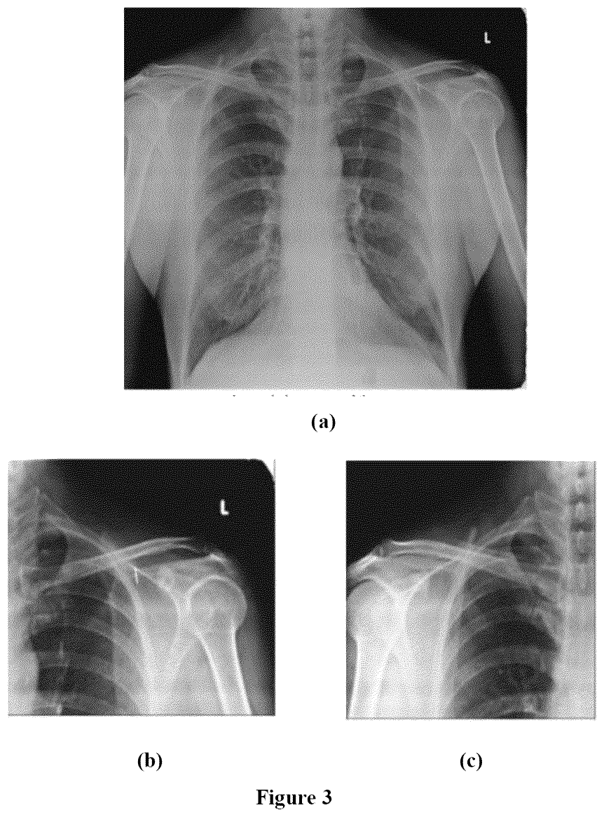

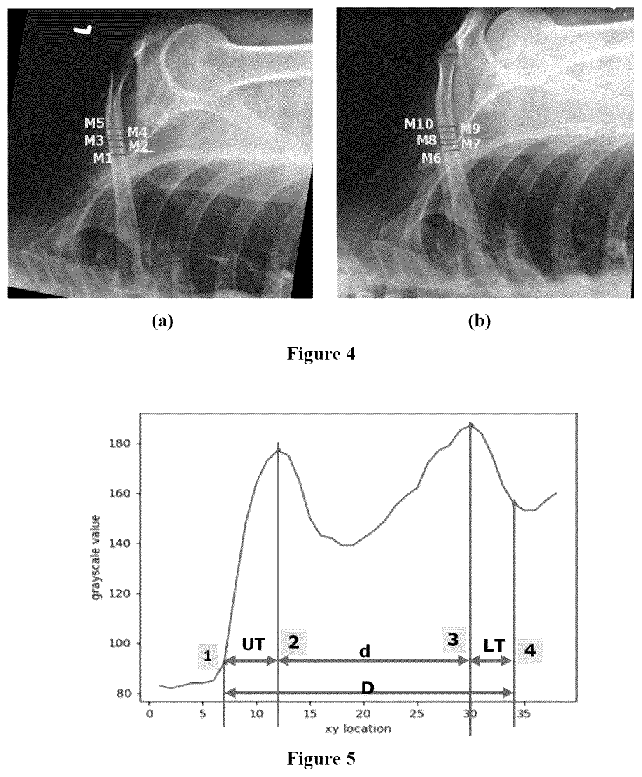

In one implementation, the modules (106) may include, for example, a receiving module (110), a determining module (111), a Bone Mineral Density (BMD) estimation module (112), a risk assessment module (113) and other modules (114). It will be appreciated that such aforementioned modules (106) may be represented as a single module or a combination of different modules. In one implementation, the data (105) may include, for example, one or more images (107) as shown in FIG. 3 (a-c), Region of Interests (ROI) (108) as shown in FIG. 4 (a-b), Bone Mineral Density (BMD) (109).

In one embodiment, the receiving module (110) is configured to receive one or more images (107) of a standard digital or computed X-ray radiograph. The one or more images (107) comprises the standard digital or computed chest X-ray radiograph as shown in FIG. 3 (a-c) for a fully automated computerized digital or computed X-ray radiographic image processing. The one or more images (107) may be in a form of a gray-scale image.

In one embodiment, the determining module (111) is configured to determine Region of Interests (ROI) (108) on the one or more images (107). The ROI (108) may be such as but not limited to bilateral clavicle bones. These bilateral clavicle bones may be masked using an Image Annotator for an automated computerized accurate segmentation of the ROI (108). Further, the determining module (111) is configured to utilize a deep neural network architecture and the deep neural network architecture is trained with a data set of images and their corresponding masks created at the ROI (108). Then, the determining module (111) performs a mapping process to segment masked region (clavicle bone) automatically from the one or more images (107) using the trained deep neural network architecture. The ROI (108) is an intersection point of the bilateral clavicle bone and an end enclosure part of a rib bone which may an ideal location for an automated digital radiographic clavicle radiogrammetry measurements, where there are no overlapping or surrounding bone structures, soft tissues, and may provide high reproducibility for the accurate automated digital or computed X-ray radiographic clavicle radiogrammetry measurements. The automated digital or computed X-ray radiographic clavicle radiogrammetry measurements is an objective type of computerized measurement and provides high accuracy.

As shown in FIG. 4 (a-b), total 10 Regions of Interests (ROI) (108) are selected for an automated digital X-ray radiographic bilateral (both left side and right side) Clavicle Radiogrammetry measurements M1 to M10. At each region, a histogram has been obtained. FIG. 5 shows an exemplary histogram for one of the regions of measurements M1-M10 of an automated digital X-ray radiographic bilateral (both left side and right side) Clavicle Radiogrammetry. From each histogram, Upper Cortical Thickness ‘UT’ (cm); Lower Cortical Thickness, ‘LT’ (cm); Endosteal Width, ‘d’ (cm); and Periosteal Width, ‘D’ (cm) all are measured in an automated way.

At ROI (108), the determining module (111) is configured to utilize a centroid region of the bilateral clavicle bone as a starting point and may proceed towards an upper direction by incrementing pixel coordinates till a white pixel is determined and the white pixel is designated as the starting point of bone (1) (also referred as bilateral clavicle bone). Further, the centroid region of the bilateral clavicle bone is progressed further by the determining module (111) in an upper direction till a black pixel is determined, that is designated as an outer point of the bilateral clavicle bone (2). The same approach is repeated in a downward direction by decrementing pixel coordinates from the centroid region of the bilateral clavicle bone, and subsequently (3) and (4) are determined.

In an embodiment, the Bone Mineral Density (BMD) estimation module (112) is configured to perform automated digital or computed X-ray radiographic clavicle radiogrammetry using determined aforementioned measurements such as 1, 2, 3, 4 to determine Endosteal width, d (cm) and Periosteal width, D (cm).

Further, the Bone Mineral Density (BMD) estimation module (112) is configured to estimate Cortical Bone Mass Indices of the Clavicle using the obtained Endosteal width, d and Periosteal width, D.

In an embodiment, the Bone Mineral Density (BMD) estimation module (112) is configured to estimate a Hip (NECK) BMD (also referred as BMD (109)) in g/cm², Hip (TOTAL) BMD (also referred as BMD (109)) in g/cm², Spine (TOTAL) BMD (also referred as BMD (109)) in g/cm², 10-year probability of Major Bones (Hip, Spine, Humerus or forearm) Osteoporotic Fracture Risk Score (%) and 10-year probability of Osteoporotic Hip Fracture Risk Score (%) using the calculated bone mass indices of the Clavicle Region of Interests (ROI) (108).

In an embodiment, the risk assessment module (113) is configured to output a risk assessment based on the calculated T-score of Hip (Neck), Hip (Total) and Spine (Total) BMD (109). The risk assessment module (113) compares the calculated T-score of Hip (Neck), Hip (Total) and Spine (Total) to a Cut-off T-score Values as per WHO's Diagnostic Criteria and predict whether the patient is normal, having osteopenia and osteoporosis. According to the WHO's Diagnostic Criteria: i) Normal (T-Score of −1 or greater), ii) Osteopenia (T-Score between −1 to −2.5), and iii) Osteoporosis (T-Score of −2.5 or less). As per the published reference paper, the threshold values for the fracture risk scores are given for the Indian Population: i) 10-year probability of Major Bones Osteoporotic Fracture Risk Score (%)≥10, and ii) 10-year probability of Osteoporotic Hip Fracture Risk Score (%)≥3. By comparing the estimated fracture risk scores with these published threshold values referred above, it determines the high future risk for osteoporotic fracture.

In an embodiment, the other modules (114) may perform a standard pre-processing on the one or more images (107). The standard pre-processing is performed using an image equalization technique to obtain the one or more images (107) with improved contrast. The following post processing techniques such as but not limited to Contrast Limited Adaptive Histogram Equalization (CLAHE), a blur, a dilation, a median filter, a scar operator, a binarization technique, connected component analysis and morphological operations, and a 2D filter is applied to the determined ROI (108). The CLAHE is applied to improve contrast of the ROI (108). The blur is applied to suppress a low intensity for all regions other than the ROI (108). The dilation is applied to increase a width of the ROI (108) with high contrast. The median filter is applied to smoothen the ROI (108) by removing low level noise. The scar operator is applied to extract the ROI (108) from the gray-scale image. The binarization technique such as an OTSU method may be applied to convert the gray-scale image to binary image. The connected component analysis and morphological operations eliminates small noise components from the ROI (108). The two-dimensional filter is applied to increase size of thin surfaces in the ROI (108) using a sharpening operator.

In an embodiment, it is inferred that in case due to same contrast between the clavicle bone and surrounding or due to scapular bone intersection, either upper bone or lower bone extraction is missing or sometimes both. In such a case, from the obtained centroid point, the determining module (111) increments or decrements to either left or right up to a specified maximum pixel position and may repeat obtaining the width measurement. Further, in case if output mask shape is wrong, then, the determining module (111) is configured to increment or decrement the pixel coordinate to either left or right up to a specified maximum pixel position and may repeat obtaining the width measurement. Further in case, if scapular bone touches the clavicle bone either in upper or lower bone region then there may be an increase in the bone width false positively. Then in such a case, the determining module (111) is configured to increment or decrement pixel coordinate to either left or right up to a specified maximum pixel position and may obtain the width measurement.

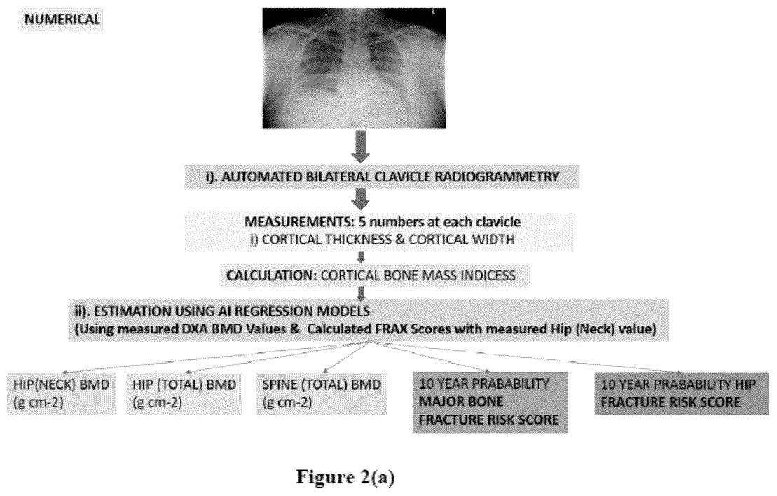

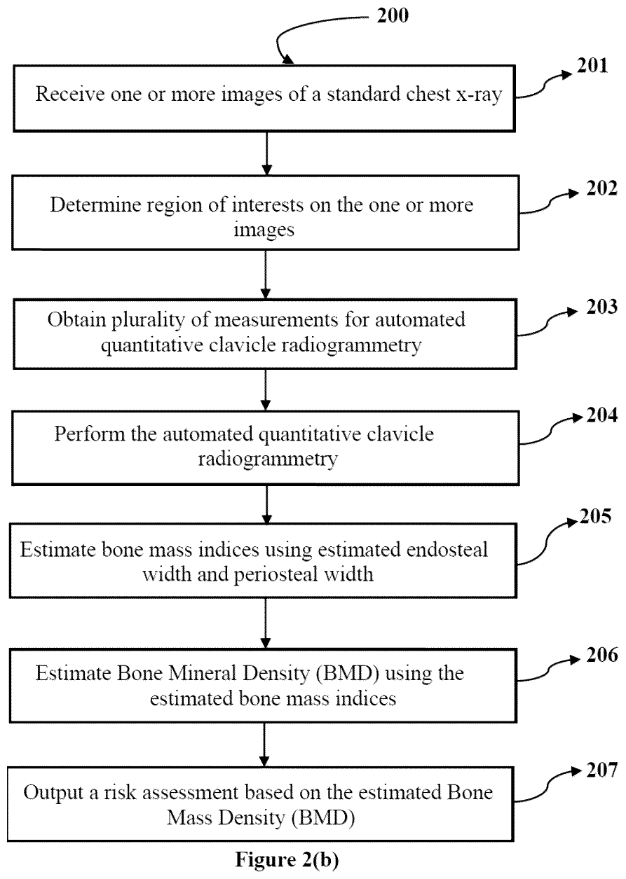

In another aspect, the present invention provides a method for bone fracture risk assessment. FIG. 2 (a) shows an exemplary flow chart illustrating the steps of numerical approach for an automated estimation of Cortical Thickness of the Clavicle and Calculation of Cortical bone mass indices of the Clavicle to predict Hip (Neck) Bone Mineral Density (BMD), Hip (Total) BMD and Spine (Total) BMD and its associated 10-year probability of Major Bone and Hip Osteoporotic Fracture Risk scores with high accuracy from a Low-cost Chest X-ray image using an automated digital or computed X-ray radiographic bilateral clavicle radiogrammetry in accordance with some embodiments of the present disclosure. In particular, FIG. 2 (b) shows an exemplary flow chart illustrating a method for performing risk assessment of bone fracture using an automated digital or computed X-ray radiographic bilateral clavicle radiogrammetry, in accordance with some embodiments of the present disclosure.

At step (201), receiving, by a receiving module (110) one or more images (107). The one or more images (107) comprises a standard digital Chest X-ray as shown in FIG. 3 (a-c) for an automated computerized image processing. The one or more images (107) may be in the form of a gray-scale image.

In an embodiment, the other modules (114) are configured to perform a standard pre-processing on the one or more images (107). The standard pre-processing is performed using an image equalization technique to obtain the one or more images (107) with improved contrast.

At step (202), determining, by a determining module (111) Region of Interests (ROI) (108) on the one or more images (107). The ROI (108) may be such as but not limited to bilateral clavicle bones. These bilateral clavicle bones may be masked using an Image Annotator for an automated computerized accurate segmentation of the ROI (108) using UNet a deep neural architecture that performs semantic segmentation in a complex environment. Further, at the step (202) performing, by the other modules (114) post processing techniques, such as but not limited to CLAHE, a blur, a dilation, a median filter, a scar operator, a binarization technique, connected component analysis and morphological operations, a Two-Dimensional (2D) filter and the like on the ROI (108). The CLAHE is applied to improve contrast of the ROI (108). The blur is applied to suppress a low intensity for all regions other than the ROI (108). The dilation is applied to increase a width of the ROI (108) with high contrast. The median filter is applied to smoothen the ROI (108) by removing low level noise. The scar operator is applied to extract the ROI (108) from the gray-scale image. The binarization technique such as an OTSU method may be applied to convert the gray-scale image to a binary image. The connected component analysis and morphological operations together eliminates small noise components from the ROI (108). The two-dimensional filter is applied to enhance the edges of the ROI (108) using a sharpening operator. Further, at the step (202), training, by the determining module (111) a deep neural network architecture with a data set of images and their corresponding masks created at the ROI (108). Further at the step (202) performing, by the determining module (111) a mapping process to segment the masked region (clavicle bone) automatically from the one or more images (107) using the trained deep neural network architecture. The ROI (108) is an intersection point of the bilateral clavicle bone and an end enclosure part of a rib bone which may an ideal location for an automated digital or computed X-ray radiographic clavicle radiogrammetry measurements, where there are no overlapping or surrounding bone structures, soft tissues, and may provide high reproducibility for the accurate automated digital or computed X-ray radiographic clavicle radiogrammetry measurements.

At step (203), plurality of measurements is obtained for an accurate automated digital or computed X-ray radiographic bilateral clavicle radiogrammetry. As shown in FIG. 4 (a-b), total 10 Regions of Interests (ROI) (108) are selected for an automated bilateral (both left side and right side) Clavicle Radiogrammetry measurements M1 to M10. At each region, a histogram has been obtained. FIG. 5 shows an exemplary histogram for one of the regions of measurements M1-M10 of an automated digital X-ray radiographic bilateral (both left side and right side) Clavicle Radiogrammetry. From each histogram, Upper Cortical Thickness ‘UT’ (cm); Lower Cortical Thickness, ‘LT’ (cm); Endosteal Width, ‘d’ (cm); and Periosteal Width, ‘D’ (cm) all are measured in an automated way. Further, at the step (203), utilizing, by the determining module (111) a centroid region at the ROI (108) of the bilateral clavicle bone as a starting point and proceeding towards an upper direction by incrementing pixel coordinates till a white pixel is determined and designating the white pixel as the starting point of bone (1). Further, at the step (203), progressing, by the determining module (111) the centroid region of the bilateral clavicle bone in an upper direction till a black pixel is determined, and designating the black pixel as an outer point of the bilateral clavicle bone (2). The same approach is repeated in a downward direction by decrementing the pixel coordinates from the centroid region of the bilateral clavicle bone, and subsequently (3) and (4) are determined.

At step (204), performing, by Bone Mineral Density (BMD) estimation module (112) the automated semi-quantitative digital or computed X-ray radiographic bilateral clavicle radiogrammetry using the measurements such as 1, 2, 3, 4 to determine Endosteal width, d (cm) and Periosteal width, D (cm).

At step (205), estimating, by the Bone Mineral Density (BMD) estimation module (112) Cortical Bone Mass Indices of the Clavicle using the measured Endosteal width, d and Periosteal width, D of the Clavicle

At step (206), estimating, by the Bone Mineral Density (BMD) estimation module (112) a Hip (NECK) BMD (also referred as BMD (109)) in g/cm², Hip (TOTAL) BMD (also referred as BMD (109)) in g/cm², Spine (TOTAL) BMD (also referred as BMD (109)) in g/cm², 10-year probability of Major Bones (Hip, Spine, Humerus or forearm) Osteoporotic Fracture Risk Score (%) and 10-year probability of Osteoporotic Hip Fracture Risk Score (%) using the calculated bone mass indices of the Clavicle Region of Interests (ROI) (108).

At step (207), outputting, by a risk assessment module (113) a risk assessment based on based on calculated T-score of Hip (Neck), Hip (Total) and Spine (Total) from the corresponding estimated BMD (109) of the Hip (Neck), Hip (Total) and Spine (Total). The risk assessment module (113) compares the calculated T-score of Hip (Neck), Hip (Total), and Spine (Total) to a cut-off T-score values as per WHO's Diagnostic Criteria and predict whether the patient is normal, having osteopenia and osteoporosis. According to the WHO's Diagnostic Criteria: i) Normal (T-Score of −1 or greater), ii) Osteopenia (T-Score between −1 to −2.5), and iii) Osteoporosis (T-Score of −2.5 or less). As per the published reference paper, the threshold values for the fracture risk scores are given for the Indian Population: i) 10-year probability of Osteoporotic Hip Fracture Risk Score (%)≥3. By comparing the estimated fracture risk scores with these published threshold values referred above, it determines the high future risk for osteoporotic fracture.

In above-mentioned aspect of the present invention, the invention discloses a numerical approach for an automated estimation of Cortical Thickness of the Clavicle and Calculation of Cortical bone mass indices of the Clavicle to predict dual Hips (Neck) Bone Mineral Density (BMD), dual Hips (Total) BMD and Spine (Total) BMD and its associated 10-year probability of Major Bone and Hip Osteoporotic Fracture Risk scores with high accuracy from a Low-cost Chest X-ray image using an automated digital X-ray radiographic bilateral clavicle radiogrammetry. The present disclosure assesses the following parameters using the radiograph in an automated way with good accuracy: i) calculated Bone Mass Indices of the clavicle bone; and ii) estimated areal BMD (g/cm2) which may show statistically significant correlations with measured areal BMD by DXA, an expensive ‘gold’ standard technique, referring example 1. In an exemplary embodiment, the sensitivity and specificity of the empirical formula earlier may be utilized to estimate areal hip BMD using the calculated clavicle bone mass indices from the chest radiograph. The result is found to be 82% and 94% respectively, when compared to a Hip BMD value, measured by a standard Dual Energy X-ray Absorptiometry (DXA) bone densitometer. The estimated total hip BMD using the empirical formula is correlated with a total hip BMD (Hologic make) for a total studied population and is statistically significant (r=0.88, P<0.01). The empirical formula is identified as better tool for bone risk assessment for total population and for population of older age with a sensitivity (88.8 and 95.6%), a specificity (89.6 and 90.9%), a positive predictive value (88.8 and 95.6%) and a negative predictive value (89.6 and 90.9%), respectively.

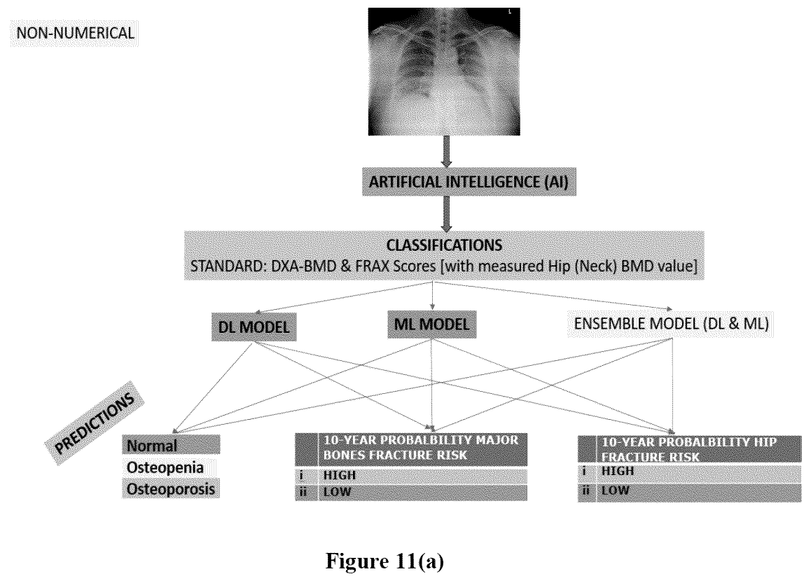

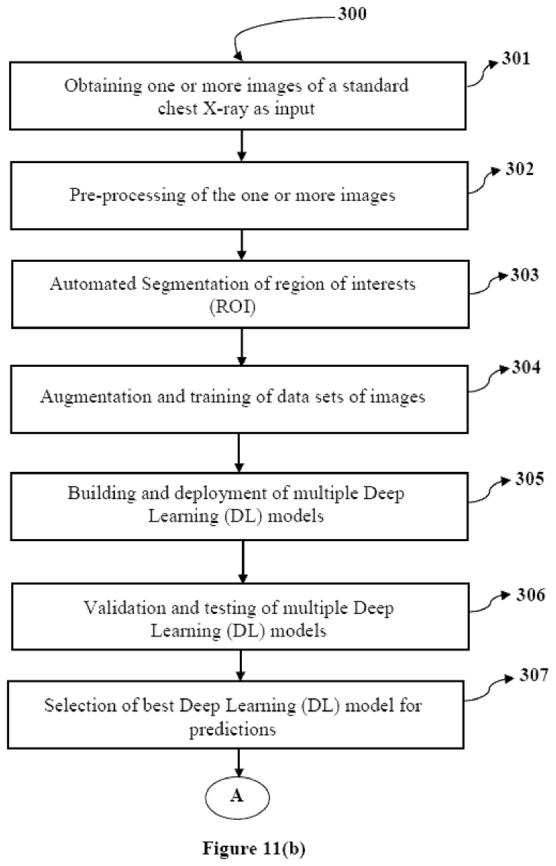

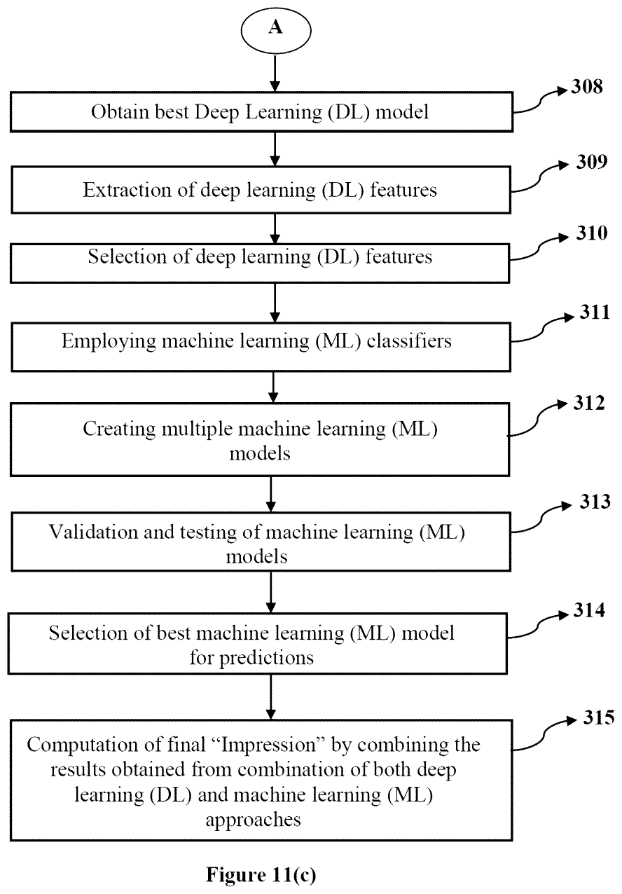

In another aspect, the present invention provides a method for bone fracture risk assessment. FIG. 11 (a) shows an exemplary flow chart illustrating the steps of non-numerical approach for an automated prediction of Osteoporosis and Osteopenia and 10-year probability of major bones (hip, spine, humerus or forearm) Osteoporotic fracture risk and hip fracture risk using Artificial Intelligence (AI) with automated classifications in machine learning (ML) model and/or deep learning (DL) model and/or ensembling model with high accuracy from a chest X-ray image. FIG. 11 (b-c) shows an exemplary flow chart (300) illustrating a method for performing risk assessment of bone fracture using Artificial Intelligence (AI) with machine learning (ML) model and/or deep learning (DL) model and/or ensembling model with high accuracy from a chest X-ray image, in accordance with some embodiments of the present disclosure.

In this aspect, the invention provides the method steps of non-numerical approach for an automated prediction of Osteoporosis and Osteopenia using Artificial Intelligence (AI) with automated classifications in machine learning (ML) model and/or deep learning (DL) model and/or ensembling model with high accuracy from a chest X-ray image.

At step (301), obtaining one or more images (107) as input by a receiving module (110). The one or more images (107) comprises a standard digital Chest X-ray as shown in FIG. 3 (a-c) for an automated computerized image processing. The one or more images (107) may be in DICOM, jpg, png or tiff format.

In an embodiment, at step (302), a standard pre-processing on the one or more images (107) is performed by the other modules (114). The standard pre-processing is performed using an image equalization technique to obtain the one or more images (107) with improved contrast. At step (302), checking the image format and if the image is in DICOM format, automatically extract patient information, including Name, Age, and Sex, and save it in the report. Further, converting, the received image into PNG format to ensure uniformity for all types of images in the subsequent procedures and verifying, if the received image is a CXR using GoogleNet classifier model, if not discarding the image and requesting an upload of a chest X-ray image. Furthermore, at step (302), resizing the image into 512×512 size and applying Contrast Limited Adaptive Histogram Equalization (CLAHE) to the resized input image.

At step (303), performing an automated segmentation of region of interests (ROI). An automated ROI segmentation is performed wherein EfficientNet B7 with UNet decoder and 40 dropout was trained and obtained the model to automatically segment the ROI (108) from the CLAHE applied image. ROI (108) is a square region on CXR which covers clavicle bone end at the left and right side, extend top up to shoulder neck interaction at the top and cover up to L2 at bottom end.

At step (304), applying, augmentation techniques to increase number of images dataset and to generalize the model performance to handle versatile images. The model is well trained and evaluated with parameters such as dice_coef, dice_loss, IoU, Recall, and Precision. Further, at step (304), extracting the grayscale image information of the automatically segmented ROI (108) by mapping ROI mask with original grayscale image.

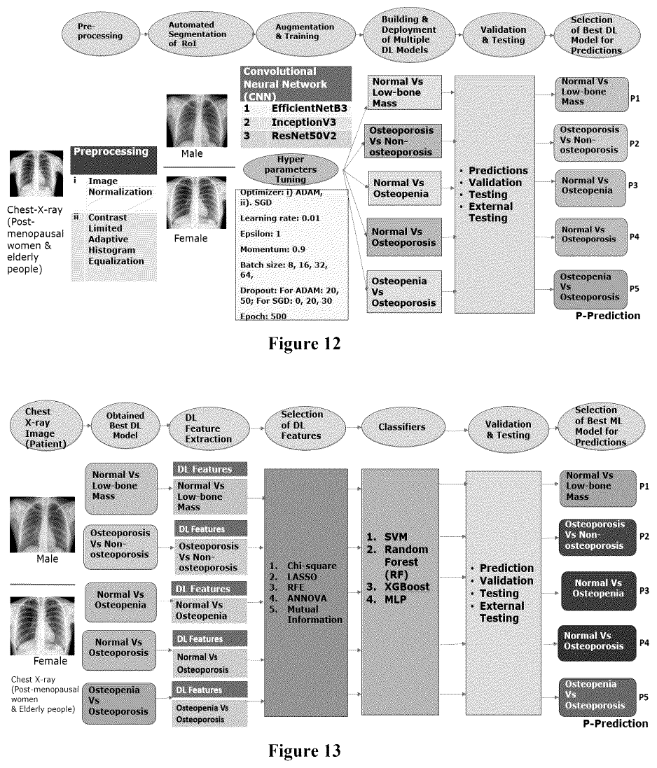

At step (305), building and deployment of multiple Deep Learning (DL) models comprises creating separate binary classification Deep Learning (DL) model for male and female to classify Osteoporosis, Osteopenia and Normal cases. The schematic framework for this DL approach is given in FIG. 12. Training the binary models to perform the classification comprises Osteoporosis vs Normal; Osteopenia vs Normal; Osteoporosis vs Osteopenia; Low Bone Mass vs Normal and Osteoporosis vs Non-Osteoporosis. At step (305), choosing the different deep learning models to perform automatic classification task such as EfficientNetB3, Inception V3 and ResNet50V2 and training each model to classify all categories with different hyperparameters combinations. Again, applying, augmentation techniques to increase number of images dataset and to generalize the classification model performance to handle versatile input images. In this work, augmentation iteration=3 are applied to create class balanced dataset for training, testing and validation dataset with various techniques.

At step (306), validating and testing of multiple Deep Learning (DL) models trained with augmented dataset and validated with parameters comprising accuracy, loss, AUC, precision, recall.

At step (307), selecting the best model in each binary classification category based on the model's validation and test data performance. Deep Learning (DL) classification results are computed by the five predictions comprising Low bone mass vs Normal; Osteoporosis vs non-Osteoporosis; Normal vs Osteopenia; Normal vs Osteoporosis; and Osteopenia vs Osteoporosis.

At step (308), obtaining the best Deep Learning (DL) model.

At step (309), extracting deep features from the penultimate layer (dense layer) of the best Deep Learning (DL) model by giving the output of step (302) for the deep feature extraction method is provided.

At step (310), selection of Deep Learning (DL) features is performed by applying different feature selection techniques to the datasets.

At step (311), employing various Machine Learning (ML) classifiers with various combination of feature selection techniques is performed.

At step (312), creating the separate machine learning (ML) models for the different categories, all the machine learning (ML) models are trained with all combinations of feature selection techniques. The schematic framework for this ML approach is given in FIG. 13.

At step (313), validating and testing of multiple machine learning (ML) models.

At step (314), selecting the best model of each category based on the performance of model on the validation and test dataset. Machine Learning (DL) classification results are computed by the five predictions comprising Low bone mass vs Normal; Osteoporosis vs non-Osteoporosis; Normal vs Osteopenia; Normal vs Osteoporosis; and

Osteopenia vs Osteoporosis.

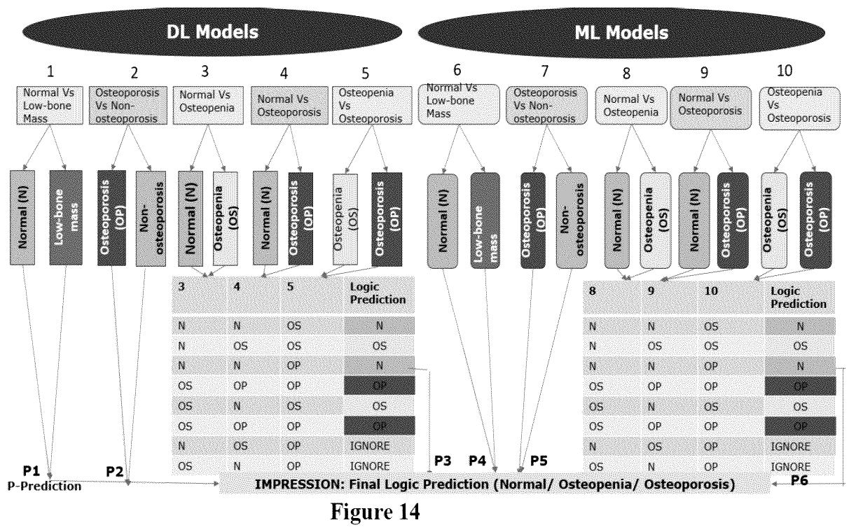

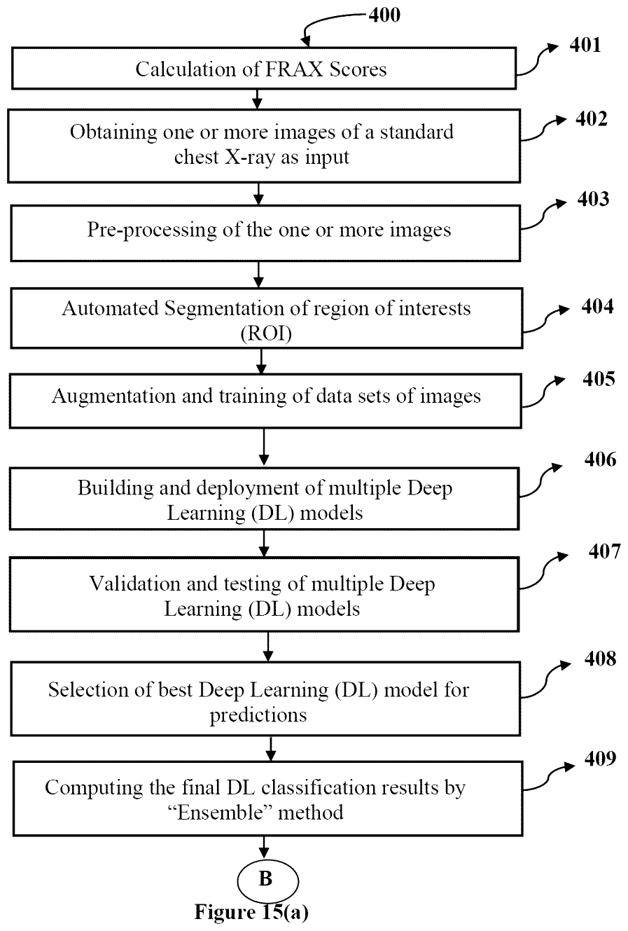

At step (315), computing final “Impression” by ensembling the results obtained from combination of both deep learning (DL) and machine learning (ML) approaches. The schematic framework for the combination of both DL and ML approaches is shown in FIG. 14.

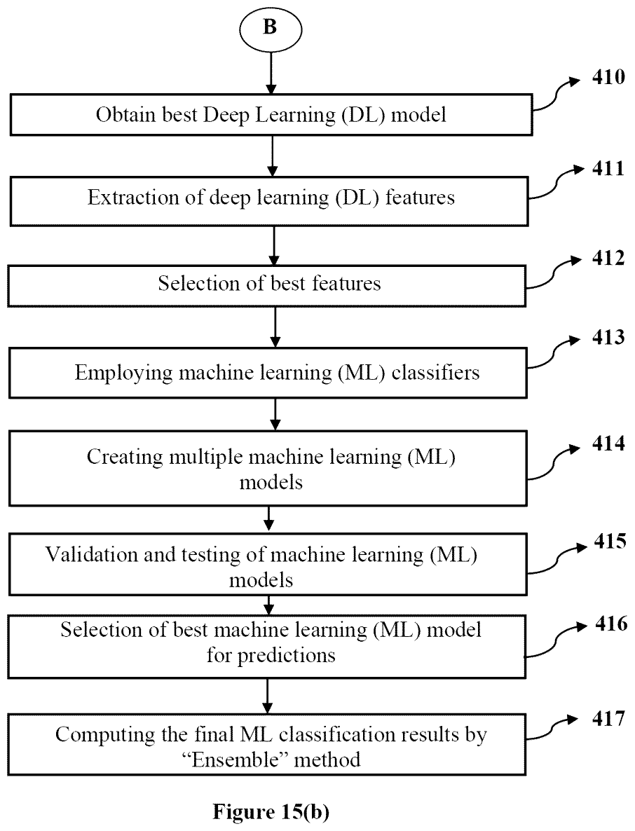

In another aspect, the present invention provides a method for bone fracture risk assessment. FIG. 15 (a-b) shows an exemplary flow chart (400) illustrating a method for performing risk assessment of bone fracture using a bone fracture risk assessment system with deep learning (DL) and machine learning (ML) approaches for prediction of 10-year probability of Major Bone and Hip Osteoporotic Fracture Risk from a conventional low-cost chest X-ray image, in accordance with some embodiments of the present disclosure.

In particular, in this aspect, the present invention provides a method for prediction of the following fracture risk from a low-cost conventional Chest X-ray image with good accuracy, compared to the calculated FRAX Scores with measured Hip (Neck) BMD by DXA using the Online FRAX Tool for Indian Population as standard:
i) 10-year Probability of Major Bones (Hip, Spine, Humerus or Forearm) Osteoporotic Fracture Risk (FRAX Score≥10%) with Hip (Neck) BMD, measured by DXA of Hologic Type
ii) 10-year Probability of Hip Fracture Risk (FRAX Score≥3%) with Hip (Neck) BMD, measured by DXA of Hologic Type

At step (401), the FRAX scores are calculated for selected group of patients. In particular, at step (401), for each study patient, the following clinical investigations were done: i) Dual Hip (both left- and right-side hips) Bone mineral density (BMD) by Dual energy X-ray Absorptiometry (DXA) of Hologic Type Machine; ii) Lumbar Spine BMD by DXA Standard; iii) Chest Posterior to Anterior View X-ray; and iv) Calculation of FRAX Scores: Using the Online Free FRAX Tool for the Indian Population, following FRAX Scores were calculated for each patient by substituting patient's measured Hip (Neck) BMD by DXA (Hologic type) and their clinical risk factors: a) 10-Year Probability of Major Bones Osteoporotic Fracture Risk Score (%) and b) 10-Year Probability of Osteoporotic Hip Fracture Risk Score (%).

At step (402), obtaining one or more images (107) as input by a receiving module (110). The one or more images (107) comprises a standard digital Chest X-ray as shown in FIG. 3 (a-c) for an automated computerized image processing. The one or more images (107) may be in DICOM, jpg, png or tiff format.

In an embodiment, at step (403), a standard pre-processing on the one or more images (107) is performed by the other modules (114). The standard pre-processing is performed using an image equalization technique to obtain the one or more images (107) with improved contrast. At step (403), checking the image format and if the image is in DICOM format, automatically extract patient information, including Name, Age, and Sex, and save it in the report. Further, converting, the received image into PNG format to ensure uniformity for all types of images in the subsequent procedures and verifying, if the received image is a CXR using GoogleNet classifier model, if not discarding the image and requesting an upload of a chest X-ray image. Furthermore, at step (403), resizing the image into 512×512 size and applying Contrast Limited Adaptive Histogram Equalization (CLAHE) to the resized input image.

At step (404), performing an automated segmentation of region of interests (ROI). An automated ROI segmentation is performed wherein EfficientNet B7 with Unet decoder and 40 dropout was trained and obtained the model to automatically segment the ROI (108) from the CLAHE applied image. ROI (108) is a square region on chest X-ray which covers clavicle bone end at the left and right side, extend to top up to the patient's shoulder, neck interaction at the top and cover up to the Lumbar Spine L2 at bottom end.

At step (405), applying, augmentation techniques to increase number of images dataset and to generalize the model performance to handle versatile images. The model is well trained and evaluated with parameters such as dice_coef, dice_loss, IoU, Recall, and Precision. Further, at step (405), extracting the grayscale image information of the automatically segmented ROI (108) by mapping ROI mask with original grayscale image.

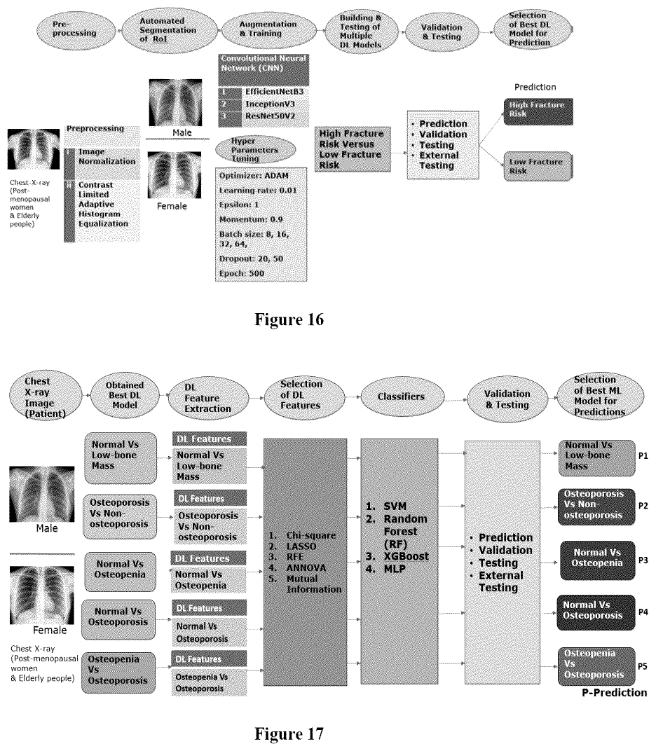

At step (406), building and deployment of multiple Deep Learning (DL) models is provided. The schematic framework for this DL approach is given in the FIG. 16. A single binary classification Deep Learning (DL) models are trained using the Chest X-ray Image dataset of the patients to be studied to classify the individual who is at the high risk for future osteoporotic fracture using the chest X-ray image. Deep Learning (DL) models comprise the following:

### FRAX Score (FX)-1:10-Year Probability of Major Bones Osteoporotic Fracture Risk Score (%)

i. Label-1: Those who are having High Risk Threshold for 10-Year Probability of Major Bones Osteoporotic Fracture (Score≥10%)
ii. Label-2: Those who are having Low Risk for 10-Year Probability of Major Bones Osteoporotic Fracture (Score<10%)

### FRAX Score (FX)-2:10-Year Probability of Osteoporotic Hip Fracture Risk Score (%)

i. Label-1: Those who are having High Risk Threshold for 10-Year Probability of Osteoporotic Hip Fracture (Score≥3%)
ii. Label-2: Those who are having Low Risk for 10-Year Probability of Osteoporotic Hip Fracture (Score<3%)

Also, at step (406), choosing the different deep learning models to perform automatic classification task such as EfficientNetB3 and InceptionV3 and training each model to classify all categories with different hyperparameters combinations. Again, applying, augmentation techniques to increase number of images dataset and to generalize the classification model performance to handle versatile input images. In this work, augmentation iteration=3 are applied to create class balanced dataset for training, testing and validation dataset with various techniques.

At step (407), validating and testing of multiple Deep Learning (DL) models trained with augmented dataset and validated with parameters comprising accuracy, loss, AUC, precision, recall.

At step (408), selecting the best Deep Learning (DL) model in each binary classification category based on the model's validation and test data performance.

At step (409), Deep Learning (DL) classification results are computed by “Ensemble” method. An Ensemble DL Model is created for the following:
i. FX-1:10-Year Probability of Major Bones Osteoporotic Fracture Risk Score (≥10)
ii. FX-2:10-Year Probability of Osteoporotic Hip Fracture Risk Score (≥3).

Further, at step (409), for Major Bone Osteoporotic Fracture Risk, selection of Best “3” DL Models of FX-1 and testing of all these models with input images get from the steps (404, 405) are provided. Each model provides that's binary label as output and finally maximum number of time repeated label is considered as the final label output for Major Bone Osteoporotic Fracture Risk. Furthermore, at step (409), for Osteoporotic Hip Fracture Risk, selection of Best “3” DL Models of FX-2 and testing of all these models with input images get from the steps (404, 405) are provided. Each model provides that's binary label as output and finally maximum number of time repeated label is considered as the final label output for Osteoporotic Hip Fracture Risk.

At step (410), the best Deep Learning (DL) model is obtained.

At step (411), extracting deep features from the penultimate layer (dense layer) of the best Deep Learning (DL) model developed earlier by giving the output of step (403) for the deep feature extraction method is provided.

At step (412), selection of best features is performed by applying different feature selection techniques to the datasets.

At step (413), employing various Machine Learning (ML) classifiers with various combination of feature selection techniques is performed.

At step (414), creating the separate machine learning (ML) models for the FX-1 and FX-2 categories with entire datasets. All the ML models have been trained with all combinations of feature selection techniques. The schematic framework for this ML approach is given in FIG. 17.

At step (415), validating and testing of multiple machine learning (ML) models.

At step (416), selecting the best model of each category based on the performance of model on the validation and test dataset.

At step (417), Machine Learning (DL) classification results are computed by “Ensemble” method. Separate Ensemble model created for the following:
FRAX Score (FX)-1: Major Bones Osteoporotic Fracture Risk Scores (≥10%)
FRAX Score (FX)-2: Osteoporotic Hip Fracture Risk Scores (≥3%)

Further, at step (417), for Major Bone Osteoporotic Fracture Risk, selection of Best “3” of ML Models of FX-1 is provided. All these models tested with input features which are extracted from the penultimate layer of the corresponding FX-1 categories best DL model. Each model provides that's binary label as output and finally maximum number of time repeated label is considered as the final output label (FX-1) for Major Bone Osteoporotic Fracture Risk. Furthermore, at step (417), for Osteoporotic Hip Fracture Risk, selection of Best “3” of ML Models of FX-2 is provided. All these models tested with input features which are extracted from the penultimate layer of the corresponding FX-2 categories best DL model. Each model provides that's binary label as output and finally maximum number of time repeated label is considered as the final output label (FX-2) for Osteoporotic Hip Fracture Risk.

Present disclosure relates to an Artificial Intelligence (AI) with machine learning or deep learning techniques-based tool indigenously configured to estimate bilateral hip BMD by an automatic way using the digital or computed chest radiograph which may be tested at multisite centres across country, where there may be a high incidence of osteoporosis in local population, exorbitant healthcare costs or limited access to state-of-the art imaging centres.

The present disclosure shall be installed on a computer connected to any diagnostic X-ray machine and may serve as a low-cost “screening tool” to predict future osteoporotic fracture risk at hip and other major bones accurately in the risk group population such that, customized therapeutic intervention may be administered for the individual in order to prevent further bone mineral loss.

Moreover, in any Hospitals, chest X-ray is taken for any patient as a first basic investigation in a general health check-up. The present disclosure does not require any additional test to estimate hip BMD and uses recently taken chest radiograph or X-ray of the patients for the estimation of the hip BMD. Hence, the present disclosure is cost effective as compared to conventional techniques.

The embodiments described in the above-detailed description of the present invention are explained in detail with reference to the following examples comprising the study of patients for predicting Osteoporosis and Osteopenia and its associated fracture risk automatically with high accuracy from a conventional chest X-ray image using novel innovative approaches using “iOsteoporos Screen” i.e., system and method for bone fracture risk assessment of the present invention.

### Subjects (Patients)

A hospital-based screening for osteoporosis and its associated fracture risk was conducted in a total of 1300 post-menopausal women and aged people of both females and males. It had an institutional ethical committee approval from Chettinad Academy of Research and Education, Chettinad Hospital, Chennai. The defined inclusion—as well as exclusion-criteria for the study participants were given as follows. The following, who had given informed written consent for this study was included: i). Post-menopausal woman, ii). Both female & male, aged 50 years and above, and iii). Known cases of osteoporosis, with and without a previous fracture. According to the exclusion criteria, the participants of this study were free from any chronic illness of thyroid, liver, kidney and heart; those who had any major organ (kidney, liver, heart) transplantation and accident-induced fractures, and who took therapeutic drugs for bone and its complications were excluded; further, participant with the following was excluded: pacemaker, defibrillator, vertebral internal fixations, bone cement, other foreign metal object in the chest region, abnormal chest imaging, pneumonia, abnormal lung lesions, an artifact in chest image and low-quality chest image.

### Methods

### i) Dual Energy X-Ray Absorptiometry (DXA) Bone Densitometer

The areal bone mineral density (BMD) in gcm⁻² at both left- and right-sides of the hip (bilateral hips) and lumbar spine (anterior to posterior) of each participant was measured by a standard DXA bone densitometer (Make: Hologic) using a standard protocol by a well-trained and certified radiographer.

### ii) Digital Chest X-Ray (CXR)

A standard digital chest posterior-to-anterior (PA) view X-ray (CXR) was taken in all the participants using a digital X-ray machine with an X-ray tube voltage and tube current of 90-95 kVp and 6-8 mAs, respectively, at film tube distance of 180 cm.

### Measurements & Classifications

### 1. DXA

The bone mineral density (BMD) at following regions of interests (ROI) was measured quantitatively by the dedicated software provided by the DXA bone densitometer manufacturer: i) Hip Neck region (N-BMD) (both left- and right-sides), ii) Hip Total region (T-BMD) (both left- and right-sides), iii) Lumbar spine (L): Average L1 to L4 (L-BMD). As the automated diagnostic report given by the DXA machine was based on Asian's BMD Reference Data base, the published values of ICMR (Indian Council of Medical Research)'s Indian Sex-specific Normal BMD Data was used in this study to calculate T-score. Then, the participant was classified into following groups by the lowest calculated T-score of dual Hips (Neck), dual Hips (Total) and Spine (Total) according to the WHO's Diagnostic Criteria: i) Normal (T-Score of −1 or greater), ii) Osteopenia (T-Score between −1 to −2.5), and iii) Osteoporosis (T-Score of −2.5 or less).

### 2. Estimation of FRAX Score Using Online FRAX Tool

For each participant, FRAX Score (Fracture Risk Assessment Tool) was calculated using its Online FRAX Tool for Indian Population, which is available at: https://frax.shef.ac.uk/FRAX/tool.aspx?country=51

The following details available in the filled-in self-prepared Questionnaire for each participant was used for estimating the FRAX Score (Fracture Risk Assessment Tool):
i. Age (in years)
ii. Sex: Female/Male
iii. Body wight (Kg)
iv. Body height (cm)
v. Previous Osteoporotic fracture: (Yes/No)
vi. Parent Fractured Hip: (Yes/No)
vii. Current Smoking: (Yes/No)
viii. Glucocorticoids: (Yes/No)
ix. Rheumatoid arthritis: (Yes/No)
x. Secondary osteoporosis: (Yes/No)
xi. Alcohol 3 or more units/day: (Yes/No)
xii. Hip (Neck) BMD (g cm⁻²): The make of DXA equipment used was selected (Hologic Type) and then the measured Hip (Neck) BMD (g cm⁻²) value was substituted.

### An Experimental Setup for an Automated Digital X-Ray Image Processing

The standard C×R image (either in DICOM or JPEG format), obtained for each participant was resized to 512×512 pixels using the following image pre-processing techniques: i). Image Normalization; and ii). Contrast Limited Adaptive Histogram Equalization. In order to perform an effective training of the image data, each image was augmented using the following techniques, namely: i). Rotation (±5°), ii). Shifting (±3%), and iii). Zoom (<15%). For this study, a standard ROIs of the image was defined as follows: The boundary for top and bottom of the image covered sixth cervical vertebrae and second lumbar vertebrae respectively; whereas the boundary for left- and right-side of the image included the complete clavicle length on both sides. The ROI of the image was segmented in an automated way using an efficient B7 network as shown in FIG. 5 (a-b).

Development of ‘iOsteoporos Screen’ (an Intelligent Osteoporosis Screening Tool)

In female and male, the normative T-scores of dual hips and spine by DXA varies significantly. Also, in the C×R image of both female and male, the internal bone and its surrounding body structure at the chest region varies significantly. Due to these variations, sex-specific multiple deep learning (DL) convolutional neural network (CNN) models as well as machine learning (ML) models were developed and tested using a standard C×R image dataset of the participants with labels defined by BMD measured by DXA based diagnosis of osteoporosis and osteopenia (lumbar spine, hip (neck), or hip (Total) T-score≤−2.5) as Standard in a supervised learning manner.

In order to predict 10-year probability of Osteoporotic Hip Fracture Risk form the C×R image, both DL and ML models were developed with labels defined by FRAX Score, calculated for the Indian population using the Online FRAX Tool as Standard in a supervised learning manner.

The developed DL/ML Model is an artificial intelligence (AI)-powered Software Tool, named and Trademark registered as ‘iOsteoporos Screen’ (an Intelligent Osteoporosis Screening Tool); it can predict osteoporosis & osteopenia and its associated osteoporotic fracture risk in the risk group of population from the C×R image with high accuracy, compared to T-score of measured dual Hips (Neck) BMD or N-BMD, dual Hips (Total) BMD or T-BMD and Spine (Total) BMD or L-BMD by DXA, as standard. The CXR dataset of the participants were divided into female and male separately. It was selected and split by 80%, 10% and 10% automatically for the following: i). training, ii). validation, and iii). internal test respectively.

### Example 1

An Automated Estimation of Cortical Thickness of the Clavicle and Calculation of Cortical Bone Mass Indices of the Clavicle to Predict Dual Hips (Neck) Bone Mineral Density (BMD), Dual Hips (Total) BMD and Spine (Total) BMD and its Associated 10-Year Probability of Major Bones and Hip Osteoporotic Fracture Risk Scores with High Accuracy from a Low-Cost Chest X-Ray Image

An automated estimation of Cortical thickness of the Clavicle and calculation of Cortical bone mass indices of the Clavicle is performed on the two bases:
(i) When compared to Hip BMD and Spine BMD measured by DXA as Standard:
(ii) When compared to FRAX Score, calculated from the Online FRAX Tool with the measured Hip (Neck) BMD by DXA (Hologic Machine) as Standard:

The steps for the said automated estimation process are as below:
1. Get Input: Input image (m×n) size. [Coronal view Chest PA view X-Ray image]
2. If DICOM image: Automatically extract patient's name, age and gender information from the X-ray image and then save it in the Excel file.
3. Convert DICOM to PNG image: Hounsfield unit to Grayscale conversion (pixel intensity 0 to 255).
4. Apply Histogram equalization and Median filter:
   (a) Reduce artefacts and ensures image smoothing.
   (b) Improves contrast.
5. Image size Normalization: Resized to 1024×1024 size.
6. Inspect the input image is Coronal view chest X-Ray image or not: GoogLeNet classifier ensures the input image is coronal view of chest X-Ray image.
7. Split the image into 4 quadrants, each 512×512 in size:
   (a) 1^{st} contains Right Clavicle Bone (ROI).
   (b) 2^{nd} contains Left Clavicle Bone (ROI).
   (c) 3^{rd} and 4^{th} Quadrants were eliminated.
8. Separate Left and Right-side clavicle quadrants for bone width analysis.
9. Left clavicle width measurement:
   (a) Consider the image's 2^{nd} quadrant which contains left clavicle bone.
   (b) Automatically find and extraction the Mask of Clavicle bone region (ROI) after the rib using Deep learning segmentation technique.
   (c) Find the centre points of mask (entire bone's centre point vertically).
   (d) Form a line by Connecting the first and last point.
   (e) Calculate clavicle bone's elevation angle from bottom horizon (ang).
   (f) Rotate mask image and Left side grayscale image for the angle (90-ang).
      (i) This step alliances the clavicle bone vertically.
      (ii) Helps uniform perpendicular bone width measurement.
   (g) Again, find the centre points for the rotated Mask's, horizontal extreme points and Mask length.
   (h) Histogram Analysis for Perpendicular width measurement (Bottom to top approach) as shown in FIG. 5 (a-b):
      (i) Located ROI after rib i.e., bottom of the rotated clavicle mask.
      (ii) From the bottom point, 15 pixel was left [y_max−15 vertically] to avoid wrong measurement due to rib cage cure structure.
      (iii) Start width measurement at the 16^{th} pixel, find two points (x_min−5, y_max−15) and (x_max+5, y_max−15) horizontally.
         a. x-min is x-axis value at the where mask intensity changes from (0 to 1) which means background to bone.
         b. x-max is x-axis value at the where mask intensity changes from (1 to 0) which means bone to background
         c. −5 and +5 pixels helps to start and end the measurement with some background region.
      (iv) Obtain histogram from clavicle vertical axis between above mentioned points.
      (v) Detect four points from clavicle vertically top to bottom:
         a. Point 1: Transition from background to bone (abrupt intensity rise).
         b. Point 2: The point where the First half maximum intensity (First peak) starts to decrease.
         c. Point 3: The point again intensity reaches the Second maximum intensity (Second peak).
         d. Point 4: Transition from bone to background (abrupt intensity reduce).
      (vi) Endosteal width or Inner width (d) of the clavicle bone is measured in cm by computing pixel difference between point 2 and point 3 then divided by 27.
      (vii) Periosteal width or Outer width (D) of the clavicle bone is measured in cm by computing pixel difference between point 1 and point 4 then divided by 27.
      (viii) Upper Thickness (UT) of the clavicle is measured in cm by computing pixel difference between points 1 and point 2 then divided by 27
      (ix) Lower Thickness (LT) of the clavicle is measured in cm by computing pixel difference between point 3 and point 4 then divided by 27.
      (x) Following Cortical Bone Mass Indices of the Clavicle were computed:
         a) Combined Cortical Thickness of the Clavicle=(D-d) cm
         b) Percentage Combined Cortical Thickness of the Clavicle=[(D−d/D)*100]
      (xi) If any of these 4 points not obtained, first x_min and x_max expands horizontally by 3 pixels to cover the bone region completely.
      (xii) Even after done the above step, if 4 points not obtained, next give upwards vertical shift by 3 pixels, continue step iv to ix to get width measurement.
      (xiii) Repeat step (i to x) for 5 times with a 3-pixel decrement in y axis (1 mm difference)
         a. Help to Obtain 5 Clavicle Radiogrammetry Measurements at 5 Different Locations of the Clavicle.
      (xiv) Finally Mean values are computed from the measurement analysis.
10. Right-side Clavicle Radiogrammetry Measurement:
   a. Create horizontal Flip for Quadrant 1 (right-side clavicle)
   b. Follow the above same steps to measure the Right-side Clavicle bone d, UT, LT and D width.
11. Average Width Measurement
   a. Average Upper Thickness (UT in cm) of the Clavicle is computed by Avg UT=[(MUT (left side)+MUT (right side))/2]
   b. Average Lower Thickness (LT in cm) of the Clavicle is computed by Avg LT=[(MLT (left side)+MLT (right side))/2]
   c. Average Combined Cortical Thickness of the Clavicle (D−d in cm) is computed by Avg (D−d)=([M (D−d) (left side)+M (D−d) (right side))/2]
   d. Average Percentage Combined Cortical Thickness of the Clavicle is computed by Avg [(D−d)/D*100]={M (D−d/D*100) (left side)+M (D−d/D*100) (right side))/2}
   e. The above mentioned measured/calculated average values of the clavicle have been utilized as input for an automatic regression model to predict the following with high correlation coefficients:
   I). When compared to Hip and Spine BMD measured by DXA as Standard:
   i). Score-1: Prediction of Hip (Neck) Bone Mineral Density (BMD)
   ii) Score-2: Prediction of Hip (Total) BMD (it includes the BMD of the following regions of the hip: Neck, Trochanteric, Inter-trochanteric, and Ward's triangle)
   iii) Score-3: Prediction of Spine (Total) BMD (it includes the BMD of the following regions of the Lumbar Spine (LS: LS1, LS2, LS3 and LS4)
   II). When compared to FRAX Score, calculated from the Online FRAX Tool with the measured Hip (Neck) BMD by DXA (Hologic Machine) as Standard:
   iv). Score-4:10-year probability of Major Bones Osteoporotic Fracture Risk Score (%)
   v) Score-5:10-year probability of Osteoporotic Hip Fracture Risk Score (%)
      Development of an Automated Regression Models to Predict Scores 1 to 5 with Correlation Coefficients Using an Automated Digital X-Ray Radiographic Clavicle Radiogrammetry
f. Three different regression models have been developed to predict the Scores (labels) 1, 2, 3, 4 and 5.
i. Artificial Neural Network (ANN)
ii. Pattern Recognition Neural Network (PRNN)
iii. Radial Basis Function Neural Network (RBFNN)
g. Input: The above-mentioned regression models consists of the following Clavicle Radiogrammetry values measured from the Chest X-ray Image as input variables:
i. average UT (cm),
ii. average LT (cm),
iii. average D−d (cm)
iv. average [(D−d/D)*100] values.
Output: The following Standard Score (either Score-1, Score-2, Score-3, Score-4 or Score-5) is used as an output.
h. The training input variables are generated by computing the average UT, average LT, average (D−d), and average [(D−d/D)*100] values for all input chest X-ray Images.
i. The training input variables, along with their corresponding scores are saved in an Excel file which is used during the training of the regression model.

### 1) Artificial Neural Network (ANN) Regression Model:

(a) Multiple ANN Regression Model have been created based on ANN Architecture:
(b) The Neural Network model has been built using TensorFlow's Sequential class.
(c) Here we have used four layers.
   i. The first layer consists of 160 hidden units/neurons with the ReLU activation function. ReLU stands for Rectified Linear Units.
   ii. The second layer consists of 480 hidden units with the ReLU activation function.
   iii. The third layer consists of 256 hidden units with the ReLU activation function.
   iv. The final layer is the output layer which consists of one unit with a linear activation function.
(d) Used the Mean Squared Logarithmic Loss as loss function, and Adam loss function optimizer.
(e) Considering different combination of input variables:
   i. Initially, models were created with each input variable considered separately (Avg UT, Avg LT, Avg D-d and Avg D-d/D)
   ii. Various combinations of input variables were also explored (Avg UT, Avg LT, Avg D-d and Avg D-d/D)
   iii. The above-mentioned input combination is trained and tested for 5 different score (labels). This label combination also created a greater number of ANN models.
(f) Hyper parameter tuning such as batch size, dropout rate, learning rate and number of hidden units of each layer also created a greater number of ANN models
   a). Model validation metrics are:
      a. Mean Squared Error: {MSE}
      b. Correlation between predicted value and actual label: {correlation}
      c. R-squared (R2) Score: {R2}
   b). Graphs plotted:
      a. Scatter Plot
      b. Residual Plot
      c. Distribution of Residuals
      d. Quantile-Quantile (QQ) Plot

### 2) Pattern Recognition Neural Network (PRNN) Regression Model:

a. PRNN architecture has two hidden layers, each with “number of hidden units” neurons using the ReLU activation function. The output layer has a single neuron, as it is a regression task.
b. Choosing “Number of hidden units” is the hyperparameter tuning involved in the PRNN model
c. Number of models created is based on similar way as explained in ANN model
d. Model validation metrics are:
   i. Mean Squared Error: {MSE}
   ii. Correlation between predicted value and actual label: {correlation}
   iii. R-squared (R2) Score: {R2}
e. Graphs plotted:
   i. Scatter Plot
   ii. Residual Plot
   iii. Distribution of Residuals
   iv. Quantile-Quantile (QQ) Plot

### 3) Radial Basis Function Neural Network (RBFNN) Regression Model:

a. RBFNN has input neuron corresponding to the number of clusters obtained from KMeans. This model directly uses the RBF activations as input for the Linear Regression model. The output layer has a single neuron, as it is a regression task. The model is trained using the RBF activations as input and the actual target values (‘Score’) as the output.
b. Number of models created is based on similar way as explained in ANN model
c. “Number of Cluster” is the hyperparameter to tune and find the best one.
d. Model validation metrics are:
   i. Mean Squared Error: {MSE}
   ii. Correlation between predicted value and actual label: {correlation}
   iii. R-squared (R2) Score: {R2}
e. Graphs plotted:
   i. Scatter Plot
   ii. Residual Plot
   iii. Distribution of Residuals
   iv. Quantile-Quantile (QQ) Plot

### Clinical Application:

i. Diagnosis of Osteoporosis at Hip and Spine:
The predicted values of the following can be used to estimate the T-score of dual Hips (Neck), dual Hips (Total), and Spine (Total) of the individual studied; then based on the WHO's diagnostic Criteria, the individual can be diagnosed as Osteoporosis or Osteopenia or Normal.
   a. Predicted value of Hip (Neck) Bone Mineral Density (BMD)
   b. Predicted value of Hip (Total) BMD
   c. Predicted value of Spine (Total) BMD
      ii. Prediction of FRAX Score (Future Osteoporotic Fracture Risk) with Hip (Neck) BMD Measured by DXA (Hologic Machine):
The predicted values of the following FRAX Scores with Hip (neck) BMD measured by DXA (Hologic machine) can be used to estimate following risk for future osteoporotic fracture from the low-cost Chest X-ray Image with high accuracy:
   a. 10-year probability of Major Bones Osteoporotic Fracture Risk Score (%)
   b. 10-year probability of Osteoporotic Hip Fracture Risk Score (%)

### 1. Mean Value Calculation:

a) Finding the sum of absolute differences of that element with all other elements. (https://www.geeksforgeeks.org/array-formed-using-sum-of-absolute-differences-of-that-element-with-all-other-elements/?ref=rp)
b) The elements with Minimum absolute differences reflect they all are having nearly equal values.
c) The cluster the elements:
   i. For the UT, LT and D-d parameters, we considered the Minimum absolute differences is equal to Minimum absolute differences+0.2. This help to include all the closer value elements.
   ii. For ((D−d)/D)*100), we considered the Minimum absolute differences is equal to Minimum absolute differences+0.1.
   iii. Form the element array by mapping the index of the Minimum absolute difference's values.
d) Mean value=sum of each elements/total number of elements in the cluster.
e) Finally Mean values of the following are computed for both sides of the Clavicle separately:
   i. UT cm
   ii. LT cm
   iii. (D−d) cm

$$iv . [ ( D - d ) / D ) * 100 ) ]$$

### 2. Dataset:

Total Numbers: 1227 Chest X-ray Images of the Studied Population (Post-menopausal women and Elderly people) (933 Images are in DICOM Format & 294 Images are in jpg format)

### 3. Data Augmentation:

i. Rotation_range=5%, #Random rotation between 0 and 5 degree
ii. Width_shift_range=[−3,3]
iii. Height_shift_range=[−3,3]
iv. Zoom_range=0.15
v. Brightness_range=[0.2,0.9]
vi. Horizontal_flip=True
vii. Fill_mode=‘constant’, cval=0)
   Total Numbers of Chest X-Ray Image after Augmentation: 36000

### 4. Segmentation:

a) Automatic clavicle bone segmentation, here the ROI is Clavicle bone after ribs and till clavicle end
b) We use 80% of the data as the train set, the remaining 20% as the validation set and used unseen 303 images as test set
c) We selected the recent deep learning segmentation model i.e., EfficientNet B7 for the automatic segmentation task
d) To evaluate the model performance, the model was trained and tested with different conditions (i.e., various range of dropout rate, batch size, with and without augmentation) which is shown in the table.
e) From this table, the EfficientNet B7 with 20% dropout, batch size is 8 and with augmentation technique obtained the highest accuracy than other combination for the training, validation and testing dataset.
f) This model achieved Dice similarity co-efficient value of 0.98, IoU of 0.962, recall of 98.14%, precision of 98.0% and F1-Score of 98.0% for the training set.
g) For the validation set, the obtained Dice similarity co-efficient is 0.946, IoU of 0.9, 94.04% of recall, 95.4% precision and 94.7% of F1-Score.
h) For testing set, this model achieved Dice similarity co-efficient is 0.889, IoU of 0.807, 88.75% of recall, 90.34% precision and 89.53% of F1-Score.
i) The EfficientNet B7 with 20% dropout and with augmentation outperforms than other combination. Hence in work this model has been used for clavicle bone segmentation.

### 5. Metrics:

a. Segmentation: Performance evaluation metrices are:
   i. Dice similarity co-efficient (DSC)
   ii. Jaccard index (IoU)
   iii. Recall
   iv. Precision
   V. F-Score
b. Model evaluation [Regression Model]:
   i. Mean Squared Error (MSE):
   The Mean Squared Error measures the average squared difference between predicted and actual values. It quantifies the average squared deviation between predicted and actual values.

$$MSE = ( 1 / n ) * Σ ( y\_i - \hat{y} \_i ) \hat{} 2$$

Where:
   n is the number of data points.
   yi represents the actual value.
   ŷ_i represents the predicted value.
ii. Correlation between Predicted Value and Actual Label:
   The correlation coefficient measures the strength and direction of a linear relationship between two variables. In this case, it quantifies how well the predicted values align with the actual labels.

$$Correlation = Cov ( y , \hat{y} ) / ( σ ( y ) * σ ( \hat{y} ) ) ,$$

Where:
Cov represents the covariance between actual values y and predicted values ŷ.
σ(y) and σ(ŷ) are the standard deviations of y and ý respectively.
iii. R-squared (R2) Score:
   The R-squared score represents the proportion of the variance in the dependent variable (actual label) that is predictable from the independent variable (predicted value).

$$R \hat{} 2 = 1 - ( Σ ( y\_i - \hat{y} \_i ) \hat{} 2 / Σ ( y\_i - \overline{y} ) \hat{} 2 )$$

Where:
ý is the mean of the actual values y.
yi represents the actual value.
ŷ_i represents the predicted value.
These formulas provide a quantitative measure of the model's performance and its ability to accurately predict the target variable.

### Graphs Details [Regression Model]:

a) Scatter Plot:
   i. Purpose: The scatter plot is used to visualize the relationship between the actual target values and the predicted values from the regression model.
   ii. Justification: It helps in assessing how well the model's predictions align with the actual data points. A strong positive correlation between actual and predicted values is indicative of a well-performing model.
b) Residual Plot:
   i. Purpose: The residual plot shows the relationship between the predicted values and the residuals (the differences between actual and predicted values).
   ii. Justification: This plot is important for checking whether there are patterns or trends in the residuals. Ideally, the residuals should be randomly distributed around zero, indicating that the model is capturing the underlying patterns in the data.
c) Distribution of Residuals:
   i. Purpose: This plot displays the frequency distribution of the residuals.
   ii. Justification: It helps in assessing whether the residuals follow a normal distribution. A symmetric, bell-shaped distribution indicates that the assumptions of linear regression are met.
d) QQ Plot (Quantile-Quantile Plot):
   i. Purpose: The QQ plot is used to compare the distribution of residuals against a theoretical normal distribution.
   ii. Justification: It provides a visual assessment of how closely the residuals follow a normal distribution. A straight line indicates that the residuals are normally distributed.

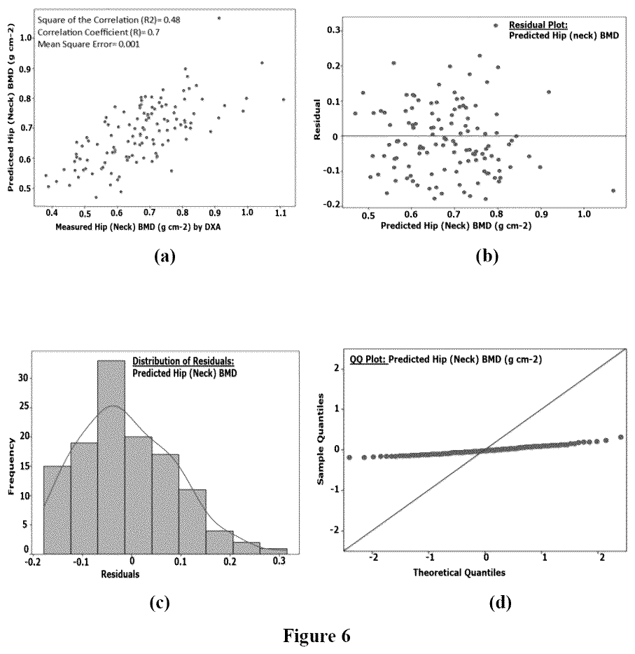

FIG. 6 (a-d) shows comparative graphs for Score 1: prediction of Hip (Neck) bone mineral density (BMD) using an automated digital X-ray radiographic clavicle radiogrammetry (from a low-cost chest x-ray image) using an artificial neural network (ANN) regression model with high correlation coefficient: (a)-Regression Model: Actual vs. Predicted; (b)-Residual Plot; (c)-Distribution of Residuals; and (d)-QQ Plot.

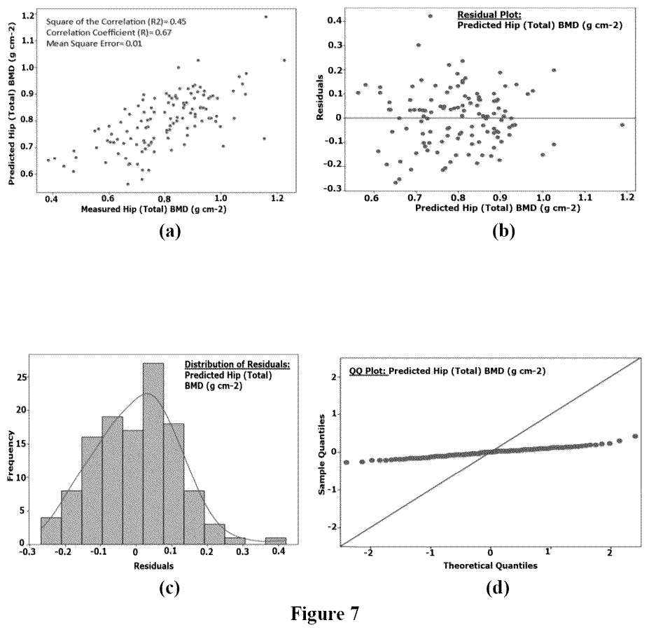

FIG. 7 (a-d) shows comparative graphs for Score 2: prediction of hip (total) bone mineral density (BMD) using an automated digital X-ray radiographic bilateral clavicle radiogrammetry (from a low-cost chest x-ray image) using a pattern recognition neural network (PNNN) regression model with high correlation coefficient: (a)-Regression Model: Actual vs. Predicted; (b)-Residual Plot; (c)-Distribution of Residuals; and (d)—QQ Plot.

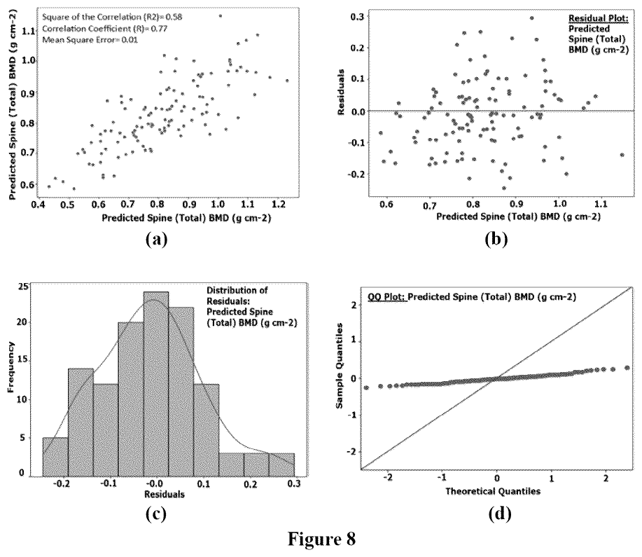

FIG. 8 (a-d) shows comparative graphs for Score 3: prediction of spine (total) bone mineral density (BMD) using an automated digital X-ray radiographic bilateral clavicle radiogrammetry (from a low-cost chest x-ray image) using an artificial neural network (ANN) regression model with high correlation coefficient: (a)-Regression Model: Actual vs. Predicted; (b)-Residual Plot; (c)-Distribution of Residuals; and (d)-QQ Plot.

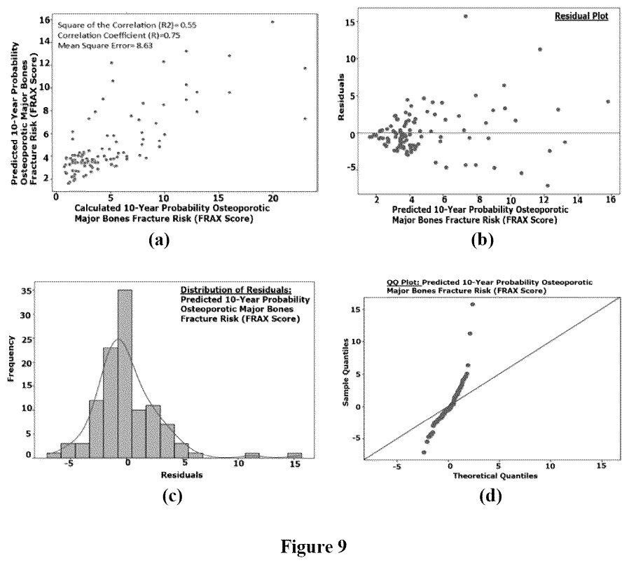

FIG. 9 (a-d) shows comparative graphs for Score 4: prediction of 10-year probability of Major Bones Osteoporotic Fracture Risk Score (FRAX Score) from an automated digital X-ray radiographic bilateral clavicle radiogrammetry (from a low-cost chest x-ray image) using pattern recognition neural network (PRNN) regression model with high correlation coefficient: (a)-Regression Model: Actual vs. Predicted; (b)-Residual Plot; (c)-Distribution of Residuals; and (d)-QQ Plot.

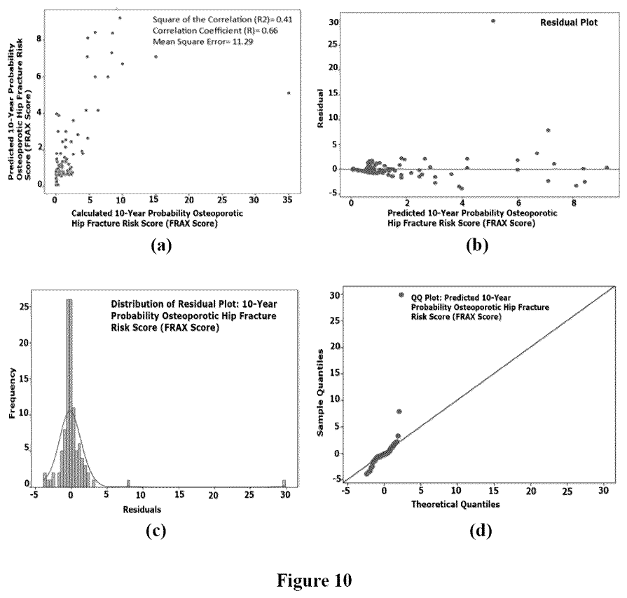

FIG. 10 (a-d) shows comparative graphs for Score 5: prediction of 10-year probability of Osteoporotic Hip Fracture Risk Score (FRAX Score) from an automated digital X-ray radiographic bilateral clavicle radiogrammetry (from a low-cost chest x-ray image) using radial basis neural network (RBRM) regression model with high correlation coefficient: (a)-Regression Model: Actual vs. Predicted; (b)-Residual Plot; (c)-Distribution of Residuals; and (d)-QQ Plot.

TABLE 1
Performance Comparison: -

| | | | | | Training Metrics | | | | Validation Metrics | | | |
|---|---|---|---|---|---|---|---|---|---|---|---|---|
| | Input parameters | | | | Dice | | | | Dice | | | |
| Sl. | Drop- | Augmen- | Batch | Learning | Coeffi- | | Preci- | F1- | Coeffi- | | Preci- | F1- |
| No. | out | tation | size | Rate | cient | Recall | sion | score | cient | Recall | sion | score |
| 1 | 0.3 | No | 8 | 0.00001 | 0.989 | 0.994 | 0.99 | 0.989 | 0.861 | 0.84 | 0.884 | 0.861 |
| 2 | 0.3 | No | 8 | 0.00001 | 0.992 | 0.996 | 0.99 | 0.992 | 0.862 | 0.837 | 0.886 | 0.861 |
| 3 | 0.4 | No | 8 | 0.00001 | 0.991 | 0.995 | 0.99 | 0.991 | 0.839 | 0.799 | 0.881 | 0.838 |
| 4 | 0.2 | No | 8 | 0.00001 | 0.991 | 0.995 | 0.99 | 0.990 | 0.861 | 0.832 | 0.887 | 0.859 |
| 5 | 0.2 | No | 8 | 0.00001 | 0.991 | 0.988 | 1.00 | 0.991 | 0.863 | 0.864 | 0.863 | 0.864 |
| 6 | 0.2 | YES | 8 | 0.00001 | 0.980 | 0.980 | 0.98 | 0.980 | 0.945 | 0.946 | 0.945 | 0.945 |
| 7 | 0.2 | YES | 8 | 0.00001 | 0.981 | 0.981 | 0.98 | 0.981 | 0.947 | 0.940 | 0.955 | 0.948 |

| | Input parameters | | | | Test Metrics | | | |
|---|---|---|---|---|---|---|---|---|
| Sl. | | | Batch | Learning | Dice | | | F1- |
| No. | Dropout | Augmentation | size | Rate | Coefficient | Recall | Precision | score |
| 1 | 0.3 | No | 8 | 0.00001 | 0.874 | 0.877 | 0.886 | 0.881 |
| 2 | 0.2 | YES | 8 | 0.00001 | 0.890 | 0.888 | 0.903 | 0.895 |

### Summary of Results:

Table 2a: Total Number of Regression Model Developed and Tested to Predict BMD at Hip (Neck), Hip (Total) & Spine (Total) and 10-year Probability of Osteoporotic Fracture Risk Scores for Major Bones and Hip with high correlation coefficients from an automated digital X-ray radiographic bilateral Clavicle Radiogrammetry Measurements (made from Low-cost Chest-Xray Image), comparing to BMD by DXA and the calculated Online FRAX Tool as Standards.

TABLE 2a

| | | | | | Prediction of | |
|---|---|---|---|---|---|---|
| | | | | | 10-Year | |
| | | | | | probability | |
| | | | | | of Osteoporotic | |
| | | | | | Fracture Risk | |
| | | | | | Scores (%) | |
| | | | | | Major | |
| | | | | | Bones | Hip |
| | Types of | | | | Fracture | Fracture |
| | Regression | Prediction of BMD | | | Risk | Risk |
| S. | Models- | Hip | Hip | Spine | Score | Score |
| No. | Networks | (Neck) | (Total) | (Total) | (%) | (%) |
| 1. | Artificial | 30 | 30 | 30 | 30 | 30 |
| | Neural | | | | | |
| | Networks | | | | | |
| | (ANN) | | | | | |
| 2. | RBFNN | 30 | 30 | 30 | 30 | 30 |
| | (Radial | | | | | |
| | Basis | | | | | |
| | Neural | | | | | |
| | Networks) | | | | | |
| 3. | Pattern | 30 | 30 | 30 | 30 | 30 |
| | Recognition | | | | | |
| | Neural | | | | | |
| | Networks | | | | | |
| | (PRNN) | | | | | |

| | | | | | |
|---|---|---|---|---|---|
| Total Regressions Model | 90 | 90 | 90 | 90 | 90 |
| Developed & Tested | | | | | |

Note:
i. BMD: Bone Mineral Density
ii. Hip (Total) BMD includes the BMD of the following regions of the hip: Neck, Trochanteric, Inter-trochanteric, and Ward's triangle
iii. Spine (Total) BMD includes the BMD of the following regions of the Lumbar Spine (LS: LS1, LS2, LS3 and LS4)

Table 2b: Best Regression Model Developed and Tested to Predict BMD at Hip & Spine and 10-year Probability Osteoporotic Fracture Risk Scores with high correlation coefficients from an automated digital X-ray radiographic bilateral Clavicle Radiogrammetry Measurements (made from Low-cost Chest-Xray Image), comparing to BMD measured by DXA and the calculated FRAX Scores with the measured Hip (Neck) BMD by DXA using the Online FRAX Score Tool as Standards Estimated Cortical Bone Mass Indices of Clavicle from digital X-ray radiographic bilateral Clavicle Radiogrammetry Measurements:
X1=Average Upper Cortical Thickness (UT) of Clavicle
X2=Average Lower Cortical Thickness (LT) of Clavicle
X3=Average Combined Cortical Thickness (D−d) of Clavicle
X4=Average Percentage Combined Cortical Thickness [(D−d)/D*100)] of Clavicle

TABLE 2b

| | Cortical | | Selected optimum hyperparameter | | | | | | | |
|---|---|---|---|---|---|---|---|---|---|---|
| | Bone | | | | | | | Square | | |
| | mass | Neural | | | | | | of the | | |
| | Indices | Networks | | | | | | Correlation | Correlation | Mean |
| | of the | Regression | | learning | hidden | hidden | cluster | Coefficient | Coefficient | Squared |
| Prediction | Clavicle | Models | Dropout | rate | units 1 | unit 2 | size | (R2) | (R) | Error |
| Hip (Neck) | X2 and | ANN | 20 | 0.001 | 160 | 480 | — | 0.47245 | 0.692752 | 0.0097 |
| BMD | X3 | (Adam) | | | | | | | | |
| Hip (Total) | X3 | PRNN | — | — | 1, 00, | — | — | 0.448031 | 0.669677 | 0.01394 |
| BMD | | | | | 100 | | | | | |
| Spine (Total) | X1, X3 | ANN | 20 | 0.001 | 160 | 480 | — | 0.577446 | 0.772134 | 0.012 |
| BMD | | (Adam) | | | | | | | | |
| 10-Year | X1, X2, | PRNN | — | — | 50, 30 | — | — | 0.550025 | 0.752211 | 8.62867 |
| Probability of | X3 | | | | | | | | | |
| Major Bones | | | | | | | | | | |
| Osteoporotic | | | | | | | | | | |
| Fracture | | | | | | | | | | |
| Risk Score | | | | | | | | | | |
| (FRAX Score) | | | | | | | | | | |
| 10-Year | X3 | PRNN | — | — | — | 10, | — | 0.3832983 | 0.633182 | 10.2182 |
| Probability of | | | | | | 001, | | | | |
| Osteoporotic | | | | | | 000 | | | | |
| Hip Fracture | | | | | | | | | | |
| Risk Score | | | | | | | | | | |
| (FRAX Score) | | | | | | | | | | |

### Example 2

An Automated Prediction of Osteoporosis and Osteopenia from Conventional Chest X-Ray Image with High Accuracy Using “iOsteoporos Screen” Tool

### Technical Details:

1. Obtain a C×R image as input. It may be in DICOM, jpg, png, or tiff format.
2). Check the image format, If the image is in DICOM format, automatically extract patient information, including Name, Age, and Sex, and save it in the report.
3). Convert the received image into PNG format to ensure uniformity for all types of images in the subsequent procedures.
4). Verify if the received image is a CXR using GoogleNet classifier model. If not, please discard the image and request an upload of a chest X-ray image.
5). Resize image into 512×512 size.
6). CLAHE is applied to the resized input image.
7). An Automated ROI segmentation:
   a. EfficientNet B7 with Unet decoder and 40 dropout was trained and obtained the model to automatically segment the ROI from the CLAHE applied image.
   b. ROI is a square region on CXR which covers clavicle bone end at the left and right side, extend to top up to shoulder neck interaction at the top and cover up to L2 at bottom end.
   c. Augmentation techniques are applied to increase number of images dataset and to generalize the model performance to handle versatile images. In this work, augmentation iteration=3 has been applied with following techniques
      i. Rotation randomly applied between −5 or +5
      ii. Horizontal flip randomly
      iii. Wight and height shift applied between −3 or +3
      iv. Zoom in and zoom out applied between −15 or +15
      v. Brightness varied from 0.2 to 0.9
   d. The model is well trained and evaluated with parameters such as dice_coef, dice_loss, IoU, Recall, and Precision.
8). Extract the grayscale image information of the automatically segmented ROI by mapping ROI mask with original grayscale image.
9). Deep Learning (DL) Classifier: [Pre-processing and training]: The schematic framework for this DL approach is given in the FIG. 12.
   a. Created separate binary classification Deep Learning (DL) model for male and female to classify Osteoporosis, Osteopenia and Normal cases.
   b. Totally 10 models were created, 5 for Female and 5 for Male.
   c. Binary Models has been trained to perform the following classification:
      i. OSTEOPOROSIS vs NORMAL
      ii. OSTEOPENIA vs NORMAL
      iii. OSTEOPOROSIS vs OSTEOPENIA
      iv. LOW BONE MASS vs NORMAL
      v. OSTEOPOROSIS vs NON-OSTEOPOROSIS
   d. The different Deep learning models were chosen to perform automatic classification task such as EfficientNetB3, InceptionV3 and ResNet50V2
   e. Each model has been trained to classify all categories with different hyperparameters combinations. The hyperparameters are
      i. Learning rate tuned from 10-2 to 10-8
      ii. Used 20% and 50% Dropout rate
      iii. Batch size's tuned from 8 to 64
      iv. Optimizer: Adam
   f. Augmentation techniques are applied to increase number of images dataset and to generalize the classification model performance to handle versatile input images. In this work, augmentation iteration=3 has been applied to create class balanced dataset for training, testing and validation dataset with following techniques
      i. Rotation randomly applied between −5 or +5
      ii. Horizontal flip randomly
      iii. Wight and height shift applied between −3 or +3
      iv. Zoom in and zoom out applied between −15 or +15
      v. Brightness varied from 0.2 to 0.9
   g. The DL models were trained with augmented dataset and validated with flowing parameters
      i. Accuracy
      ii. Loss
      iii. AUC
      iv. Precision
      v. Recall
   h. i). Table 3a shows the total number of DL Models developed and tested using different networks and hyperparameters to predict osteoporosis and osteopenia at hip and spine in female population with high accuracy;

TABLE 3a
Number of DL Models Developed & Tested Using Different Networks &
Hyperparameters for Prediction of Osteoporosis and Osteopenia at hip
and spine in Female with high accuracy.
Optimizer: ADAM Hyperparameters: i) Learning rate (0.01), ii) Epsilon (1), iii)
Momentum (0.9), iv) Batch size (8, 16, 32, 64), v) Dropout (20, 50), vi) Epoch (500)

| | | Types of Binary Models | | | | |
|---|---|---|---|---|---|---|
| | | Normal Vs | Osteoporosis | | | |
| | | Low-bone | Vs Non- | Normal Vs | Normal Vs | Osteopenia Vs |
| | Network | Mass | Osteoporosis | Osteopenia | osteoporosis | Osteoporosis |
| 1 | EfficientNetB3 | 16 | 2 | 7 | 9 | 7 |
| 2 | InceptionV3 | 8 | 3 | 6 | 8 | 6 |
| 3 | ResNet50V2 | 6 | — | 6 | 6 | 6 |

| | | | | | |
|---|---|---|---|---|---|
| TOTAL NUMBER | 30 | 5 | 19 | 23 | 19 |
| OF DL MODELS | | | | | |
| DEVELOPED & | | | | | |
| TESTED | | | | | |

Table 3b shows the best DL Model developed and tested for each type of classification to predict osteoporosis and osteopenia at hip and spine in female population with high accuracy, compared to DXA as standard;

TABLE 3b
Best DL Model for Prediction of Osteoporosis and Osteopenia at hip and
spine in Female with high accuracy as compared to DXA as Standard

| | | | ADAM Optimizer with Selected Optimum | | | | | | | | | |
|---|---|---|---|---|---|---|---|---|---|---|---|---|
| | | | Hyperparameters | | | | | | | | | |
| | Types of | | Learning | | | | | Patients | Accuracy (%) | | | |
| S. | Binary | Best | rate | | Batch | Drop- | | (waiting | | | Internal | External |
| No. | Models | Network | (min-max) | Epsilon | Size | out | Epoch | time) | Training | Validation | Testing | Testing |
| 1 | Normal | EfficientNetB3 | 0.01 to | 1 | 32 | 50 | 500 | 50 | 91 | 76 | 68 | 76 |
| | Vs Low- | | 10⁻⁸ | | | | | | | | | |
| | bone Mass | | | | | | | | | | | |
| 2 | Osteoporosis | EfficientNetB3 | 0.01 to | 1 | 16 | 50 | 500 | 50 | 91 | 72 | 84 | 80 |
| | Vs Non- | | 10⁻⁸ | | | | | | | | | |
| | Osteoporosis | | | | | | | | | | | |
| 3 | Normal Vs | EfficientNetB3 | 0.01 to | 1 | 64 | 20 | 500 | 50 | 96.7 | 62.3 | 67.9 | 75 |
| | Osteopenia | | 10⁻⁸ | | | | | | | | | |
| 4 | Normal Vs | InceptionV3 | 0.01 to | 1 | 32 | 50 | 500 | 50 | 98 | 80 | 87 | 84 |
| | Osteoporosis | | 10⁻⁸ | | | | | | | | | |
| 5 | Osteopenia Vs | EfficientNetB3 | 0.01 to | 1 | 8 | 20 | 500 | 50 | 97 | 77 | 71 | 75 |
| | Osteoporosis | | 10⁻⁸ | | | | | | | | | |

ii). Table 4a shows the total number of DL Models developed and tested using different networks and hyperparameters to predict osteoporosis and osteopenia at hip and spine in male population with high accuracy;

TABLE 4a
Number of Developed & Tested DL Models Using Different Networks &
Hyperparameters in Male for Prediction of Osteoporosis and Osteopenia
with high accuracy.
Optimizer: ADAM Hyperparameters: i) Learning rate (0.01), ii) Epsilon (1), iii)
Momentum (0.9), iv) Batch size (8, 16, 32, 64), v) Dropout (20, 50), vi) Epoch (500)

| | | Types of Binary Models | | | | |
|---|---|---|---|---|---|---|
| | | Normal Vs | Osteoporosis | | | |
| | | Low-bone | Vs Non- | Normal Vs | Normal Vs | Osteopenia Vs |
| | Network | Mass | Osteoporosis | Osteopenia | osteoporosis | Osteoporosis |
| 1 | EfficientNetB3 | 6 | 2 | 6 | 6 | 6 |
| 2 | InceptionV3 | 6 | 4 | 6 | 6 | 6 |
| 3 | ResNet50V2 | 6 | — | 6 | 6 | 6 |

| | | | | | |
|---|---|---|---|---|---|
| TOTAL NUMBER | 18 | 6 | 18 | 18 | 18 |
| OF DL MODELS | | | | | |
| DEVELOPED & | | | | | |
| TESTED | | | | | |

Table 4b shows the best DL Model developed and tested for each type of classification to predict osteoporosis and osteopenia at hip and spine in male population with high accuracy, compared to DXA as standard;

TABLE 4b
Best DL Model for Prediction of Osteoporosis and Osteopenia in Male with
high accuracy as compared to N-BMD, T-BMD and L-BMD by DXA as Standard

| | | | ADAM Optimizer with Selected | | | | | | | | | |
|---|---|---|---|---|---|---|---|---|---|---|---|---|
| | | | Optimum Hyperparameters | | | | | | | | | |
| | Types of | | Learning | | | | | Patients | Accuracy (%) | | | |
| S. | Binary | Best | rate | | Batch | Drop- | | (waiting | | | Internal | External |
| No. | Models | Network | (min-max) | Epsilon | Size | out | Epoch | time) | Training | Validation | Testing | Testing |
| 1 | Normal Vs | EfficientNetB3 | 0.01 to | 1 | 32 | 20 | 500 | 50 | 80 | 72 | 86 | 93 |
| | Low-bone | | 10⁻⁸ | | | | | | | | | |
| | Mass | | | | | | | | | | | |
| 2 | Osteoporosis | InceptionV3 | 0.01 to | 1 | 32 | 20 | 500 | 50 | 100 | 84 | 85 | 95 |
| | Vs Non- | | 10⁻⁸ | | | | | | | | | |
| | Osteoporosis | | | | | | | | | | | |
| 3 | Normal Vs | InceptionV3 | 0.01 to | 1 | 32 | 50 | 500 | 50 | 69 | 64 | 82 | 85 |
| | Osteopenia | | 10⁻⁸ | | | | | | | | | |
| 4 | Normal Vs | EfficientNetB3 | 0.01 to | 1 | 32 | 20 | 500 | 50 | 85 | 89 | 95 | 93 |
| | Osteoporosis | | 10⁻⁸ | | | | | | | | | |
| 5 | Normal Vs | EfficientNetB3 | 0.01 to | 1 | 16 | 50 | 500 | 50 | 96 | 62 | 70 | 82 |
| | Osteoporosis | | 10⁻⁸ | | | | | | | | | |

i. The best model in each binary classification category have been chosen based on the model's validation and test data performance.
10). Check the gender (Male or Female). The output image from step 8 is used as input for the best model of each gender's binary classification model.
11). DL Classification RESULTS is computed by the following way:
   a. Low bone mass vs Normal classification model result considered as “DL_Perdition-1”
   b. Osteoporosis vs Non-Osteoporosis classification model result considered as “DL_Perdition-2”
   c. Normal Vs Osteopenia Classification model result considered as “DL_Prediction 3”
   d. Normal Vs osteoporosis Classification model result considered as “DL_Prediction 4”
   e. Osteopenia Vs Osteoporosis Classification Model result considered as “DL_Prediction 5”
12). ML classifier: [Pre-processing and Training]: The schematic framework for this ML approach is given in the FIG. 13.
   a. Extract deep features from the penultimate layer (dense layer) of the best DL model
      i. Give output of step 6 as input for the deep feature extraction method
      ii. Load the best model one by one, feed the corresponding category cases image as input
         1. Collect all the deep feature from the penultimate layer.
         2. It consists of 512 values, saved in a CSV file with its exact label at the end of the array. Finally, the array contains 513 values for each patient.
   b. Different Feature selection techniques has been applied to the dataset's to select the best ‘k’ features.
      i. Chi-square (Chi²)
      ii. Analysis of Variance (ANOVA)
      iii. Mutual information
      iv. Lasso
   c. Various Machine Learning (ML) classifiers has been employed with various combination of feature selection techniques.
      i. Support Vector Machine (SVM)
      ii. Random Forest (RF)
      iii. Multi-Layer Perceptron (MLP)
      iv. XGBoost
   d. Separate ML models have been created for the five categories in both Male and Female datasets. All the ML models have been trained with all combinations of feature selection techniques
      i). Table 5a shows the total number of ML Models developed and tested using different networks and hyperparameters to predict osteoporosis and osteopenia at hip and spine in female population with high accuracy, compared to DXA as standard;

TABLE 5a
Number of Developed & Tested ML Models Using Different Classifiers &
Features Selections Methods for Prediction of Osteoporosis and Osteopenia
in Female with high accuracy
Feature Selection Methods: i. Chi-square, ii. LASSO, iii. RFE, iv.
ANNOVA, v. Mutual Information

| | | Types of Binary Models | | | | |
|---|---|---|---|---|---|---|
| | | Normal Vs | Osteoporosis | | | |
| | | Low-bone | Vs Non- | Normal Vs | Normal Vs | Osteopenia Vs |
| | Classifiers | Mass | Osteoporosis | Osteopenia | osteoporosis | Osteoporosis |
| 1 | SVM | 18 | 6 | 18 | 18 | 18 |
| 2 | Random | 18 | 4 | 18 | 18 | 18 |
| | Forest (RF) | | | | | |
| 3 | MLP | 14 | 6 | 14 | 13 | 14 |
| 4 | XGBooster | 18 | 6 | 18 | 18 | 18 |

| | | | | | |
|---|---|---|---|---|---|
| TOTAL NUMBER | 68 | 22 | 68 | 67 | 68 |
| OF ML MODELS | | | | | |
| DEVELOPED & | | | | | |
| TESTED | | | | | |

Table 5b shows the best ML Model developed and tested for each type of classification to predict osteoporosis and osteopenia at hip and spine in female population with high accuracy, compared to DXA as standard;

TABLE 5b
Best ML Model for Prediction of Osteoporosis and Osteopenia in Female with
high accuracy as compared to N-BMD, T-BMD and L-BMD by DXA as Standard

| | Types of | | | Normalized/ | No of | |
|---|---|---|---|---|---|---|
| S. | Binary | | Feature | Non- | Selected | Accuracy |
| No. | Models | Classifier | Selection | normalized | DL Features | (%) |
| 1 | Normal Vs | Random | ANNOVA | Normalized | 20 | 70 |
| | Low-bone | Forest | | | | |
| | Mass | | | | | |
| 2 | Osteoporosis | Random | Mutual | Without | 21 | 96 |
| | Vs Non- | Forest | Information | Normalization | | |
| | Osteoporosis | | | | | |
| 3 | Normal Vs | SVM | Mutual | Normalized | 25 | 82 |
| | Osteopenia | | Information | | | |
| 4 | Normal Vs | XGBooster | ANNOVA | Without | 4 | 98 |
| | Osteoporosis | | | Normalization | | |
| 5 | Osteopenia Vs | SVM | Chi-square | Normalized | 13 | 94 |
| | Osteoporosis | | | | | |

ii). Table 6a shows the total number of ML Models developed and tested using different networks and hyperparameters to predict osteoporosis and osteopenia at hip and spine in male population with high accuracy, compared to DXA as standard;

TABLE 6a
Number of Developed & Tested ML Models Using Different Classifiers &
Features Selections Methods for Prediction of Osteoporosis, and
Osteopenia in Male with high accuracy.
Optimizer: ADAM Feature Selection: i. Chi-square, ii. LASSO, iii.
RFE, iv. ANNOVA, v. Mutual Information

| | | Types of Binary Models | | | | |
|---|---|---|---|---|---|---|
| | | Normal Vs | Osteoporosis | | | |
| | | Low-bone | Vs Non- | Normal Vs | Normal Vs | Osteopenia Vs |
| | Classifiers | Mass | Osteoporosis | Osteopenia | osteoporosis | Osteoporosis |
| 1 | SVM | 18 | 17 | 18 | 18 | 18 |
| 2 | Random | 22 | 18 | 18 | 18 | 18 |
| | Forest (RF) | | | | | |
| 3 | MLP | 14 | 14 | 14 | 14 | 14 |
| 4 | XGBooster | 14 | 14 | 14 | 14 | 14 |

| | | | | | |
|---|---|---|---|---|---|
| TOTAL NUMBER | 68 | 63 | 64 | 64 | 64 |
| OF DL MODELS | | | | | |
| DEVELOPED & | | | | | |
| TESTED | | | | | |

Table 6b shows the best ML Model developed and tested for each type of classification to predict osteoporosis and osteopenia at hip and spine in male population with high accuracy, compared to DXA as standard;

TABLE 6b
Best ML Model for Prediction of Osteoporosis and Osteopenia with high accuracy
as compared to N-BMD, T-BMD and L-BMD measured by DXA as Standard

| | Types of | | | | No of | |
|---|---|---|---|---|---|---|
| S. | Binary | | Feature | Normalized/ | Selected | |
| No. | Models | Classifier | Selection | Non-normalized | DL Features | Accuracy |
| 1 | Normal Vs | Random | Mutual | Without | 40 | 86 |
| | Low-bone | Forest | Information | Normalization | | |
| | Mass | | | | | |
| 2 | Osteoporosis | SVM | Chi-square | Without | 20 | 97 |
| | Vs Non- | | | Normalization | | |
| | Osteoporosis | | | | | |
| 3 | Normal Vs | Random | Chi-square | Normalized | 70 | 74 |
| | Osteopenia | Forest | | | | |
| 4 | Normal Vs | XGBooster | Chi-square | Without | 26 | 92 |
| | Osteoporosis | | | Normalization | | |
| 5 | Osteopenia Vs | Random | Chi-square | Without | 20 | 94 |
| | Osteoporosis | Forest | | Normalization | | |

e. The best model of each category has been selected based on the performance of the model on the validation and test dataset.
f. Performance metrics used to validate the ML models are listed below:
   i. Accuracy
   ii. Area Under the Curve (AUC)
   iii. F1-score
   iv. Sensitivity
   v. Specificity
   vi. CI_Sensitivity
   vii. CI_Specificity
13). ML Classification RESULTS are computed in a similar way to DL classification:
   a. Low bone mass vs Normal classification model result considered as “ML_Prediction-1”
   b. Osteoporosis vs Non-Osteoporosis classification model result considered as “ML_Prediction-2”
   c. Normal Vs Osteopenia Classification model result considered as “ML_Prediction 3”
   d. Normal Vs osteoporosis Classification model result considered as “ML_Prediction 4”
   e. Osteopenia Vs Osteoporosis Classification Model result considered as “ML_Prediction 5”
14). Final “Impression” Computation (Combination of both DL and ML Approaches): The schematic framework for this combination of both DL and ML approaches is given in the FIG. 14.
   a. “Both DL/ML_Prediction-3/6” were computed by combining the results obtained from the Osteoporosis vs Normal, Osteopenia Vs Normal and Osteoporosis vs Osteopenia classification models. The details explained in the Table 7.
   b. If “Normal” has a higher count than both “Osteoporosis” and “Osteopenia,” the final impression is “Normal.”
   c. If “Osteoporosis” has a higher count than both “Normal” and “Osteopenia,” the final impression is “Osteoporosis.”
   d. If “Osteopenia” has a higher count than both “Normal” and “Osteoporosis,” the final impression is “Osteopenia.”
   e. We have excluded certain results from the impression computation in order to prevent unnecessary confusion. These are:
      i. “Low Bone Mass” vs. “Normal” from the Classifier's “Low Bone Mass” result.
      ii. “Osteoporosis” vs. “Non-Osteoporosis” from the Classifier's “Non-Osteoporosis” result.

TABLE 7
Computation Logic for Prediction-3/6 in DL/ML Models

| | Probabilities of Predictions using | | | |
|---|---|---|---|---|
| | the obtained Binary DL/ ML Models | | | Final |
| | Osteoporosis vs | Osteopenia Vs | Osteoporosis vs | Prediction- |
| S. No. | Normal | Normal | Osteopenia | 3/6 |
| i. | Osteoporosis | Osteopenia | Osteoporosis | Osteoporosis |
| ii. | Osteoporosis | Osteopenia | Osteopenia | Osteopenia |
| iii. | Osteoporosis | Normal | Osteoporosis | Osteoporosis |
| iv. | Osteoporosis | Normal | Osteopenia | Nil |
| v. | Normal | Osteopenia | Osteoporosis | Nil |
| vi. | Normal | Osteopenia | Osteopenia | Osteopenia |
| vii. | Normal | Normal | Osteoporosis | Normal |
| viii. | Normal | Normal | Osteopenia | Normal |

### Example 3

Prediction of the Following Fracture Risk from a Low-Cost Conventional Chest X-Ray Image in Post-Menopausal Women and Elderly People with Good Accuracy, Compared to the Calculated FRAX Scores with Measured Hip (Neck) BMD by DXA Using the Online FRAX Tool for Indian Population as Standard:
i). 10-year Probability of Major Bones (Hip, Spine, Humerus or Forearm) Osteoporotic Fracture Risk (FRAX Score≥10%) with Hip (Neck) BMD, measured by DXA of Hologic Type
ii). 10-year Probability of Hip Fracture Risk (FRAX Score≥3%) with Hip (Neck) BMD, measured by DXA of Hologic Type

### Introduction of the Study:

This study was conducted at a Multi-specialty Hospital, Chennai. The Study Participants were post-menopausal women and elderly people. According to the inclusion- and exclusion-Criteria of the study, eligible study participant was selected.

### Clinical Investigations & Measurements:

1. A group of post-menopausal women and elderly people of both sexes were screened in a hospital for having Osteoporosis and Osteopenia, and its associated 10-year probability of Major Bones Osteoporotic Fracture Risk as well as Hip Fracture Risk.
2. For each study patient, the following clinical investigations were done:
   a. BMD by DXA Bone Densitometer: BMD at both Left- and Right-sides of hip of each participant was measured by Dual-energy X-ray Absorptiometry (DXA) bone densitometer (Make: Hologic, Model: Discovery) by a well-trained and certified radiographer. It is considered as the ‘gold’ standard for diagnosing osteoporosis/osteopenia as per WHO's diagnostic criteria
   b. Lumbar Spine BMD by DXA Standard
   c. Standard Digital Chest PA view X-ray: A standard digital chest posterior-to-anterior (PA) view X-ray was taken in all the participants using a digital X-ray machine
   d. Estimation of FRAX Score Using Online FRAX Tool: For each participant, FRAX Score was calculated using its Online Fracture Risk Assessment Tool for the is Indian Population, which available at: https://frax.shef.ac.uk/FRAX/tool.aspx?country=51. It is considered as the ‘gold’ Standard to estimate the following Osteoporotic Fracture Risk Scores:
      i). 10-year Probability of Major Bones (Hip. Spine, Humerus or Forearm) Osteoporotic Fracture Risk Score (%) with Hip (Neck) BMD, measured by DXA of Hologic Type
      ii). 10-year Probability of Hip Fracture Risk Score (%) with Hip (Neck) BMD, measured by DXA of Hologic Type
3. Standard Diagnosis:
   a. As per WHO's diagnostic Criteria, the BMD measured by DXA is considered as the ‘gold’ standard for diagnosing Osteoporosis, Osteopenia and Normal.
   b. FRAX Score is considered as the standard for estimating the future Osteoporotic Fracture Risk.
   c. In this study, following threshold values were used to identify the individual who are at high risk for future osteoporotic fracture as per the published reference papers:
      i. 10-Year Probability of Major Bones (Hip, Spine, Humerus or forearm) Osteoporotic Fracture Risk Score (%): ≥10
      ii. 10-Year Probability of Osteoporotic Hip Fracture Risk Score (%)≥3

The following details were taken from the filled-in self-prepared Questionnaire for each study population (post-menopausal women and elderly people); Also, the measured Hip (Neck) BMD by DXA (Hologic) was used.
i. Age (in years)
ii. Sex: Female/Male
iii. Body wight (Kg)
iv. Body height (cm)
v. Previous Osteoporotic fracture: (Yes/No)
vi. Parent Fractured Hip: (Yes/No)
vii. Current Smoking: (Yes/No)
viii. Glucocorticoids: (Yes/No)
ix. Rheumatoid arthritis: (Yes/No)
x. Secondary osteoporosis: (Yes/No)
xi. Alcohol 3 or more units/day: (Yes/No)
xii. Hip (Neck) BMD (g cm⁻²): Measured by DXA of Hologic Type

For each patient, the above-mentioned parameters were entered in the Online FRAX Tool, and thus the following Fracture Risk Scores were calculated:
a) 10-year Probability of Major Bones (Hip, Spine, Humerus or Forearm) Osteoporotic Fracture Risk Score with the Hip (Neck) BMD, measured by DXA of Hologic Type
b) 10-year Probability of Hip Fracture Risk Score with the Hip (Neck) BMD, measured by DXA of Hologic Type

### Published Threshold Values of Future Osteoporotic Fracture Risk for the Indian Population:

1). The Summary of the key indications for initiating anti-osteoporotic therapy: An individuals with osteopenia (T-score between −1.0 and −2.5 at the Hip (Neck or lumbar spine) with clinical risk factors [OR] 10-year probability of a hip fracture ≥3.5% based on the Online FRAX Tool (based on limited data in Indians) [OR] 10-year probability of a major bones (hip, spine, humerus or forearm) osteoporosis-related fracture ≥10.5% based on the Online FRAX Tool (based on limited data in Indians)
2). A FRAX Score predicting the 10-year probability of a hip fracture ≥3%, or a 10-year probability of a major bones (hip, spine, humerus or forearm) osteoporosis-related fracture ≥20%, is indicative of an increased risk of fracture in the future.

### Technical Details:

### 1. Clinical Study and Clinical Investigations:

a) A group of post-menopausal women and elderly people of both sexes were screened in a hospital for having Osteoporosis and Osteopenia, and its associated 10-year probability of Major Bones Osteoporotic Fracture Risk.
b) For each study patient, the following clinical investigations were done:
   i. Dual Hip (both left- and right-side hips) Bone mineral density (BMD) by Dual energy X-ray Absorptiometry (DXA) of Hologic Type Machine
   ii. Lumbar Spine BMD by DXA Standard
   iii. Chest Posterior to Anterior View X-ray
   iv. Calculation of FRAX Scores: Using the Online Free FRAX Tool for the Indian Population, following FRAX Scores were calculated for each patient by substituting patient's measured Hip (Neck) BMD by DXA (Hologic type) and their clinical risk factors:
      10-Year Probability of Major Bones Osteoporotic Fracture Risk Score (%)
      10-Year Probability of Osteoporotic Hip Fracture Risk Score (%)
2. Obtain a Chest X-ray image of the post-menopausal women and elderly population of both sexes as an input image. It may be in DICOM, jpg, png, or tiff format.
3. Check the image format, If the image is in DICOM format, automatically extract patient information, including Name, Age, and Sex, and save it in the report.
4. Convert the received image into PNG format to ensure uniformity for all types of images in the subsequent procedures.
5. Verify if the received image is a chest X-ray using GoogLeNet classifier model. If not, discard the image and request an upload of a chest X-ray image
6. Resize image into 512×512 size
7. CLAHE is applied to the resized input image
8. Automatioc ROI segmentation:
   a. EfficientNet B7 with Unet decoder and 40 dropout was trained and obtained the model to automatically segment the ROI from the CLAHE applied image.
   b. ROI is a square region on chest X-ray which covers clavicle bone end at the left and right side, extend to top up to the patient's shoulder, neck interaction at the top and cover up to the Lumbar Spine L2 at bottom end.
   c. Augmentation techniques are applied to increase number of images dataset and to generalize the model performance to handle versatile images. In this work, augmentation iteration=3 has been applied with following techniques
      i. Rotation randomly applied between −5 or +5
      ii. Horizontal flip randomly
      iii. Wight and height shift applied between −3 or +3
      iv. Zoom in and zoom out applied between −15 or +15
      v. Brightnes varied from 0.2 to 0.9
   d. The model was well trained and evaluated with parameters such as dice_coef, dice_loss, IoU, Recall, and Precision.
9. Extract the grayscale image information of the automatically segmented ROI by mapping ROI mask with original grayscale image.
10. Deep Learning Classifier: [Pre-processing and training]: The schematic framework for this DL approach is given in the FIG. 16.

A single binary classification Deep Learning (DL) models were trained using the Chest X-ray Image dataset of the post-menopausal women and elderly people of both sexes studied to classify the individual who is at the high risk for future osteoporotic fracture using the chest X-ray image.

### FRAX Score (FX)-1:10-Year Probability of Major Bones Osteoporotic Fracture Risk Score (%)

i. Label-1: Those who are having High Risk Threshold for 10-Year Probability of Major Bones Osteoporotic Fracture (Score≥10%)
ii. Label-2: Those who are having Low Risk for 10-Year Probability Major Bones Osteoporotic Fracture (Score<10%)

### FRAX Score (FX)-2:10-Year Probability of Osteoporotic Hip Fracture Risk Score (%)

i. Label-1: Those who are having High Risk Threshold for 10-Year Probability of Osteoporotic Hip Fracture (Score_3%)
ii. Label-2: Those who are having Low Risk for 10-Year Probability of Osteoporotic Hip Fracture (Score<3%)
   a. The different Deep learning models were chosen to perform automatic classification task such as EfficientNetB3 and InceptionV3
   b. Each model has been trained to classify all categories with different hyperparameters combinations. The hyperparameters are
      i. Learning rate tuned from 10-2 to 10-8
      ii. Used 20% and 50% Dropout rate
      iii. Batch size's tuned from 8 to 64
      iv. Optimizers used: Adam
   c. Augmentation techniques are applied to increase number of images dataset and to generalize the classification model performance to handle versatile input images. In this work, augmentation iteration=3 has been applied to create class balanced dataset for training, testing and validation dataset with following techniques
      i. Rotation randomly applied between −5 or +5
      ii. Horizontal flip randomly
      iii. Wight and height shift applied between −3 or +3
      iv. Zoom in and zoom out applied between −15 or +15
      v. Brightness varied from 0.2 to 0.9
   d. The DL models were trained with augmented dataset and validated with flowing parameters
      i. Accuracy
      ii. Loss
      iii. AUC
      iv. Precision
      v. Recall
   e. The best model of each binary classification category has been chosen based on the model's validation and test data performance.
11. The output image from step 8 is used as input for the best model to perform binary classification.
12. DL Classification RESULTS is computed by the following way:
   a. Final DL classification result is computed by “Ensemble” method.
13. DL Ensemble Method:
   a. An Ensemble DL Model was created for the following:
      i. FX-1:10-Year Probability of Major Bones Osteoporotic Fracture Risk Score (≥10)
      ii. FX-2:10-Year Probability of Osteoporotic Hip Fracture Risk Score (≥3)
   b. Major Osteoporotic Fracture Risk:
      i. Select Best “3” DL Models of FX-1
      ii. Test all these models with input images get from the step 8
      iii. Each model provides that's binary label as output
      iv. Finally maximum number of time repeated label is considered as the final label output.
   c. Osteoporotic Hip Fracture Risk:
      i. Select Best “3” DL Models of FX-2
      ii. Test all these models with input images get from the step 8
      iii. Each model provides that's binary label as output
      iv. Finally maximum number of time repeated label is considered as the final label output.
14. ML classifier: [Pre-processing and Training]: The schematic framework for this ML approach is given in the FIG. 17.
   a. Extract deep features from the penultimate layer (dense layer) of the best DL model developed earlier.
      i. Give output of step 6 as input for the deep feature extraction method
      ii. Load the best model one by one, feed the corresponding category cases image as input
         1. Collect all the deep feature from the penultimate layer.
         2. It consists of 512 values, saved in a CSV file with its exact label at the end of the array. Finally, the array contains 513 values for each patient.
   b. Different Feature selection techniques has been applied to the datasets to select the best ‘k’ features.
      i. Chi-square (Chi²)
      ii. Mutual information
c. Various Machine Learning (ML) classifiers has been employed with various combination of feature selection techniques.
   i. Random Forest (RF)
d. Separate ML models have been created for the FX-1 and FX-2 categories with entire datasets. All the ML models have been trained with all combinations of feature selection techniques
e. The best “3” models of each category has been selected based on the performance of the model on the validation and test dataset.
f. Performance metrics used to validate the ML models are listed below:
   i. Accuracy
   ii. Area Under the Curve (AUC)
   iii. F1-score
   iv. Sensitivity
   v. Specificity
   vi. CI_Sensitivity
   vii. CI_Specificity
15. ML Classification RESULTS is computed by the following way:
   a. Final ML classification result is computed by “Ensemble” method.
16. ML Ensemble Method:
   a. Separate Ensemble model created for the following:
   FRAX Score (FX)-1: Major Bones Osteoporotic Fracture Risk Scores (≥10%)
   FRAX Score (FX)-2: Osteoporotic Hip Fracture Risk Scores (≥3%)
   b. FX-1:
      i. Select Best “3” of ML Models of FX-1
      ii. All these models tested with input features which are extracted from the penultimate layer of the corresponding FX-1 categories best DL model.
      iii. Each model provides that's binary label as output
      iv. Finally maximum number of time repeated label is considered as the final output label (FX-1).
   c. FX-2 score:
      i. Select Best “3” of ML Models of FX-2
      ii. All these models tested with input features which are extracted from the penultimate layer of the corresponding FX-2 categories best DL model.
      iii. Each model provides that's binary label as output
      iv. Finally maximum number of time repeated label is considered as the final output label (FX-2).
17. Plot:
   a. Confusion Matrix plot
      i. Confusion matrix for trained data
      ii. Confusion matrix for tested data

Table 8a: Number of DL Models Developed & Tested Using Different Networks & Hyperparameters for Prediction of the following high threshold values of Fracture Risk Scores from a low-cost conventional Chest X-ray image in post-menopausal women and elderly people with good accuracy, compared to the calculated FRAX Scores with the measured Hip (Neck) BMD by DXA (Hologic Type) using the Online FRAX Tool for Indian Population as Standard:
i). High Risk Threshold for 10-year Probability of Major Osteoporotic Fracture (Score≥10%)
ii). High Risk Threshold for 10-year Probability of Hip Fracture (Score≥3%)

TABLE 8a
Optimizer: ADAM
Hyperparameters: i) Learning rate (0.01 to 10-8), ii) Epsilon (1), iii)
Momentum (0.9), iv) Batch size (8, 16, 32, 64), v) Dropout (20, 50),
vi) Epoch (500)

| | | Types of Binary Models Developed & | |
|---|---|---|---|
| | | Tested | |
| | | i).No Major Bones | |
| | | Osteoporotic Fracture | |
| | | Risk Vs Having 10- | ii). No Hip Fracture |
| | | year Probability of | Risk Vs Having 10- |
| | | Major Bones | year Probability of |
| | | Osteoporotic Fracture | Hip Fracture Risk |
| | | Risk Score (≥10%) | Score (≥3%) with |
| S. No. | Network | with BMD | BMD |
| 1 | Efficient Net B3 | 6 | 10 |
| 2 | Inception V3 | 6 | 10 |
| 3 | ResNet 50 V2 | 6 | Nil |

| | | |
|---|---|---|
| TOTAL NUMBER | 18 | 20 |
| OF DL MODELS | | |
| DEVELOPED & TESTED | | |

Table 8b: Best DL Models Developed & Tested Using Different Networks & Hyperparameters for Prediction of the following fracture risk from a low-cost conventional Chest X-ray image in post-menopausal women and elderly people with good accuracy, compared to the calculated FRAX Scores with measured Hip (Neck) BMD by DXA (Hologic Type) using the Online FRAX Tool for Indian Population as the Standard:
i). High Risk Threshold for 10-year Probability of Major Bones Osteoporotic Fracture (Score≥10%)
ii). High Risk Threshold for 10-year Probability of Hip Osteoporotic Fracture (Score≥3%)

TABLE 8b

| | | ADAM Optimizer with Selected | | | | | | ACCURACY (%) | |
|---|---|---|---|---|---|---|---|---|---|
| | | Optimum Hyperparameters | | | | | | | Validation |
| Types of | | Learning | | | | | Patients | | High |
| Binary | Best | rate | | Batch | Drop- | | (waiting | | Fracture |
| Model | Network | (min-max) | Epsilon | Size | out | Epoch | time) | Training | Risk |
| Low Risk for | InceptionV3 | 0.01 to 10 | 1 | 32 | 20 | 500 | 50 | 93 | 81.5 |
| Major Bones | | power −8 | | | | | | | |
| Osteoporotic | InceptionV3 | 0.01 to 10 | 1 | 16 | 20 | 500 | 50 | 95 | 92.7 |
| Fracture Vs | | power −8 | | | | | | | |
| High Risk | InceptionV3 | 0.01 to 10 | 1 | 64 | 50 | 500 | 50 | 80 | 69.2 |
| Threshold | | power −8 | | | | | | | |
| for 10-year | | | | | | | | | |
| Probability of | | | | | | | | | |
| Major Bones | | | | | | | | | |
| Osteoporotic | | | | | | | | | |
| Fracture | | | | | | | | | |
| (≥10%) | | | | | | | | | |
| Low Risk for | EfficientnetB3 | 0.01 to 10 | 1 | 64 | 20 | 500 | 50 | 76 | 92.2 |
| Hip Fracture | | power −8 | | | | | | | |
| Vs High Risk | EfficientnetB3 | 0.01 to 10 | 1 | 32 | 20 | 500 | 50 | 98 | 86.6 |
| Threshold | | power −8 | | | | | | | |
| for 10-year | EfficientnetB3 | 0.01 to 10 | 1 | 64 | 50 | 500 | 50 | 91 | 87.9 |
| Probability of | | power −8 | | | | | | | |
| Hip Fracture | | | | | | | | | |
| (≥3%) | | | | | | | | | |

| | ACCURACY (%) | | | | | | | |
|---|---|---|---|---|---|---|---|---|
| | Validation | | Testing | | | Testing (External) | | |
| Types of | Low | | High | Low | | High | Low | |
| Binary | Fracture | | Fracture | Fracture | | Fracture | Fracture | |
| Model | Risk | All | Risk | Risk | All | Risk | Risk | all |
| Low Risk for | 75 | 78 | 88.6 | 73.2 | 80 | 66 | 82 | 83 |
| Major Bones | 72.5 | 82 | 92 | 64.8 | 7 | 77 | 72 | 77 |
| Osteoporotic | 76.4 | 72 | 85.8 | 74.3 | 79 | 66 | 85 | 83 |
| Fracture Vs | | | | | | | | |
| High Risk | | | | | | | | |
| Threshold | | | | | | | | |
| for 10-year | | | | | | | | |
| Probability of | | | | | | | | |
| Major Bones | | | | | | | | |
| Osteoporotic | | | | | | | | |
| Fracture | | | | | | | | |
| (≥10%) | | | | | | | | |
| Low Risk for | 92.2 | 79 | 88.8 | 68.5 | 78.7 | 78.5 | 84.5 | 83.67 |
| Hip Fracture | 52.9 | 70 | 90.8 | 60.4 | 75 | 100 | 71 | 75 |
| Vs High Risk | 71.7 | 79 | 87.9 | 75.4 | 81 | 80 | 91 | 85 |
| Threshold | | | | | | | | |
| for 10-year | | | | | | | | |
| Probability of | | | | | | | | |
| Hip Fracture | | | | | | | | |
| (≥3%) | | | | | | | | |

Table 9a: Number of ML Models Developed & Tested for Prediction of the following fracture risk from a low-cost conventional Chest X-ray image in post-menopausal women and elderly people with good accuracy, compared to the calculated FRAX Scores with measured Hip (Neck) BMD by DXA (Hologic Type) using the Online FRAX Tool for Indian Population as standard:
i). 10-year Probability of Major Osteoporotic Fracture Risk Score (≥10%)
ii). 10-year Probability of Hip Fracture Risk Score (≥3%)

TABLE 9a
Optimizer: ADAM

| | | | Total |
|---|---|---|---|
| | | Number | Number |
| | | of ML | of ML |
| | | Models | Models |
| Types of ML Binary | Classifiers (Feature | Developed & | Developed & |
| Models | Selection method) | Tested | Tested |
| Low Risk for Major | Random Forest | 70 | 140 |
| Bones Osteoporotic | (Chi2) | | |
| Fracture Vs High | Random Forest | 70 | |
| Risk Thresholds for | (Mutual Information | | |
| 10-year | | | |
| Probability of | | | |
| Major Bones | | | |
| Osteoporotic | | | |
| Fracture | | | |
| (≥10%) | | | |
| Low Risk for | Random Forest (Chi²) | 210 | 420 |
| Osteoporotic Hip | Random Forest | 210 | |
| Fracture Vs High | (Mutual Information) | | |
| Risk Thresholds | | | |
| for 10-year | | | |
| Probability of | | | |
| Hip Fracture (≥3%) | | | |

Table 9b: Best ML Models Developed & Tested for Prediction of the following fracture risk from a low-cost conventional Chest X-ray image in post-menopausal women and elderly people with good accuracy, compared to the calculated FRAX

Scores with measured Hip (Neck) BMD by DXA using the Online FRAX Tool for Indian Population as Standard:
i). 10-year Probability of Major Bones Osteoporotic Fracture Risk Score (≥10%)
ii). 10-year Probability of Hip Osteoporotic Fracture Risk Score (≥3%)

TABLE 9b
Optimizer: ADAM

| | | | ACCURACY (%) | | | |
|---|---|---|---|---|---|---|
| | | | | Testing | | |
| | | | | | | All (Both |
| | Best ML Models | | | | | High |
| | Developed & Tested | | | | | Fracture |
| | Classifiers | No of | | High | | Risk & |
| Types of | (Feature | Selected | | Fracture | | Low |
| ML Binary | Selection | DL | | Threshold | Low | Fracture |
| Models | method) | Features | Training | Risk | Risk | Risk) |
| Low Risk for | Random | 190 | 100 | 90.5 | 88.6 | 89.5 |
| Major Bones | Forest | 200 | 100 | 95 | 88 | 91.5 |
| Osteoporotic | (Mutual | 260 | 100 | 94.7 | 90.5 | 92.6 |
| Fracture Vs | Information) | | | | | |
| High Risk | | | | | | |
| Threshold | | | | | | |
| for 10-year | | | | | | |
| Probability of | | | | | | |
| Major Bones | | | | | | |
| Osteoporotic | | | | | | |
| Fracture | | | | | | |
| (≥10%) | | | | | | |
| Low Risk for | Random | 90 | 100 | 80 | 83.3 | 81.7 |
| Hip Fracture | Forest | 170 | 100 | 86.7 | 91.1 | 88.9 |
| Vs High Risk | (Mutual | 360 | 100 | 86.7 | 88.9 | 87.8 |
| Threshold | Information) | | | | | |
| for 10-year | | | | | | |
| Probability of | | | | | | |
| Osteoporotic | | | | | | |
| Hip Fracture | | | | | | |
| (≥3%) | | | | | | |

It will be understood by those within the art that, in general, terms used herein, and are generally intended as “open” terms (e.g., the term “including” should be interpreted as “including but not limited to,” the term “having” should be interpreted as “having at least,” the term “includes” should be interpreted as “includes but is not limited to,” etc.). For example, as an aid to understanding, the detail description may contain usage of the introductory phrases “at least one” and “one or more” to introduce recitations. However, the use of such phrases should not be construed to imply that the introduction of a recitation by the indefinite articles “a” or “an” limits any particular part of description containing such introduced recitation to inventions containing only one such recitation, even when the introductory phrases “one or more” or “at least one” and indefinite articles such as “a” or “an” (e.g., “a” and/or “an” should typically be interpreted to mean “at least one” or “one or more”) are included in the recitations; the same holds true for the use of definite articles used to introduce such recitations. In addition, even if a specific part of the introduced description recitation is explicitly recited, those skilled in the art will recognize that such recitation should typically be interpreted to mean at least the recited number (e.g., the bare recitation of “two recitations,” without other modifiers, typically means at least two recitations, or two or more recitations).

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the detailed description.

1. A system (101) for bone fracture risk assessment, wherein the system (101) comprises:
a processor or a central processing unit (CPU) (102);
an Input/Output (I/O) interface (103) coupled with the processor (102); and
a memory (104) for storing instructions executable by the processor (102),
wherein the memory (104) comprises modules (106) and data (105),
wherein the data (105) comprises one or more images (107), region of interests (108) and a bone mineral density (BMD) (109), and
wherein the modules (106) comprise a receiving module (110), a determining module (111), a Bone Mineral Density (BMD) estimation module (112), a risk assessment module (113) and other modules (114).

2. The system (101) as claimed in claim 1, wherein the modules (106) are configured to perform the estimation of bone fracture risk employing the data (105).

3. The system (101) as claimed in claim 1, wherein the receiving module (110) is configured to receive the one or more images (107) of a standard digital or computed Chest X-ray radiograph.

4. The system (101) as claimed in claim 3, wherein the one or more images (107) comprises the standard digital or computed chest X-ray radiograph for a fully automated computerized digital or computed X-ray radiographic image processing and one or more images (107) is in a form of a gray-scale image.

5. The system (101) as claimed in claim 1, wherein the determining module (111) is configured to:
determine Region of Interests (ROI) (108) on the one or more images (107); and
utilize a deep neural network architecture and the deep neural network architecture is trained with a data set of images and their corresponding masks created at the Region of Interests (ROI) (108),
wherein the determining module (111) performs a mapping process to segment masked region (clavicle bone) automatically from the one or more images (107) using the trained deep neural network architecture.

6. The system (101) as claimed claim 5, wherein the Region of Interests (ROI) (108) is an intersection point of the bilateral clavicle bone and an end enclosure part of a rib bone being an ideal location for an automated digital radiographic bilateral clavicle radiogrammetry measurements.

7. The system (101) as claimed in claim 1, wherein the Bone Mineral Density (BMD) estimation module (112) is configured to:
perform automated digital or computed X-ray radiographic bilateral clavicle radiogrammetry, and
estimate a Hip (NECK) BMD (109) in g/cm2, Hip (TOTAL) BMD (109) in g/cm2, Spine (TOTAL) BMD (109) in g/cm2, 10-year probability of Major Bones (Hip, Spine, Humerus or forearm) Osteoporotic Fracture Risk Score (%) and 10-year probability of Osteoporotic Hip Fracture Risk Score (%) by employing the calculated Bone Mass Indices of the Clavicle Region of Interests (ROI) (108).

8. The system (101) as claimed in claim 1, wherein the risk assessment module (113) is configured to output a risk assessment based on calculated T-Score of dual Hips (Neck), dual Hips (Total) and Spine (Toal) from the estimated bone mineral density (BMD) (109) of the Hips (Neck), dual Hips (Total) and Spine (Toal) by comparing the estimated T-score of dual Hips (Neck), dual Hips (Total) and Spine (Toal) to a cutoff T-Score values as per World Health Organization (WHO)′ Diagnostic criteria and predicting whether the patient is Normal, having Osteopenia and Osteoporosis.

9. The system (101) as claimed in claim 1, wherein the risk assessment module (113) is configured to output a risk assessment based on estimated fracture risk scores by comparing the estimated fracture risk scores with published threshold values comprising 10-year probability of Major Bones Osteoporotic Fracture Risk Score (%)≥10 and IO-year probability of Osteoporotic Hip Fracture Risk Score (%)≥3 and determining the high future risk for Osteoporotic fracture.

10. The system (101) as claimed in claim 1, wherein the other modules (114) are configured to perform a standard pre-processing on the one or more images (107), and wherein the standard pre-processing Is performed by employing an image equalization technique to obtain the one or more images (107) with improved contrast.

11-37. (canceled)
